(19) United States
(12) Patent Application Publication
(10) Pub. No.: US 2021/0403445 A1
(43) Pub. Date: Dec. 30, 2021

(54) SYSTEMS AND METHODS FOR REGIOSELECTIVE CARBONYLATION OF 2,2-DISUBSTITUTED EPOXIDES

(71) Applicant: CORNELL UNIVERSITY, Ithaca, NY (US)

(72) Inventors: Geoffrey W. Coates, Lansing, NY (US); Aran K. Hubbell, Ithaca, NY (US)

(21) Appl. No.: 17/293,424

(22) PCT Filed: Nov. 18, 2019
(86) PCT No.: PCT/US2019/062061

§ 371 Date: May 12, 2021

Related U.S. Application Data
(60) Provisional application No. 62/768,746, filed on Nov. 16, 2018.

(51) Int. Cl. C07D 305/12; C07F 11/00; C07F 5/06; C07C 67/37; B01J 31/22; B01J 31/18

(52) U.S. Cl. CPC C07D 305/12; C07F 11/005; C07F 5/069; B01J 2231/321; B01J 31/2217; B01J 31/183; B01J 2531/31; C07C 67/37

(57) ABSTRACT

Provided are methods of carbonylating cyclic substrates to produce carbonylated cyclic products. The cyclic substrates may be 2, 2-di substituted epoxides and the cyclic products may be β,β-di substituted lactones. The method may be carried out by forming and pressurizing a reaction mixture of the cyclic substrate, a solvent, carbon monoxide, and a [LA⁺][CO(CO)4⁻] catalyst, where [LA⁺] is a Lewis acid capable of coordinating to the cyclic substrate. The method may proceed with a regio selectivity of 90:10 or greater. The resulting carbonylated cyclic products may be converted to ketone aldol products that retain the stereochemistry and enantiomeric ratio of the carbonylated cyclic products.

### CROSS REFERENCE TO RELATED APPLICATIONS

This application claims priority to U.S. Provisional Application No. 62/768,746, filed on Nov. 16, 2018, the disclosure of which is incorporated herein.

### STATEMENT REGARDING FEDERALLY SPONSORED RESEARCH

This invention was made with government support under Grant Number DE-FG02-05ER15687 awarded by the Department of Energy and Grant Number GM008500 awarded by National Institutes of Health. The government has certain rights in the invention.

### BACKGROUND OF THE DISCLOSURE

Significant progress has been made since the introduction of the Mukaiyama aldol addition (FIG. 1A), Reformatsky reaction, and other similar transformations. However, low temperatures, stoichiometric additives, and/or highly reactive α-dicarbonyl substrates are required to promote both reactivity and stereocontrol, restricting the overall scope and practicality of these transformations. Despite these drawbacks, demand is high for ketone-aldol adducts, which have proven useful for the synthesis of chiral tertiary alcohols in natural products and pharmaceuticals. Thus, alternative synthetic methods are desired.

Ketone-aldol products can also be synthesized via the cycloaddition of ketenes and ketones to produce β-lactones that are subsequently ring-opened. This method is often used in the synthesis of enantioenriched β-hydroxyacids, which are important due to their widespread use as enantiopure synthetic starting materials. However, this procedure requires low reaction temperatures to prevent ketene self-dimerization or reaction with water, and few stereoselective processes have been reported.

The resulting β-lactones are desirably versatile intermediates that can also undergo ring-opening polymerization (ROP) to produce poly(β-hydroxyalkanoate)s (PHAs). These naturally occurring, aliphatic polyesters offer a potentially biodegradable alternative to current plastics, most of which are not degradable. By tuning the architectural structure of certain PHAs, properties such as melting and degradation temperatures can be tuned to improve processability and overall practicality on an industrial scale.

The first carbonylation of isobutylene oxide, the simplest 2,2-disubstituted epoxide was made using a Co₂(CO)₈/3-hydroxypyridine catalyst. This system produced the corresponding β,β-disubstituted β-lactone in 60% yield alongside both polymer and carboxylic acid byproducts (FIG. 7). The carbonylation of isobutylene oxide was not revisited until 2002 when we reported a method that afforded a 4:1 ratio of β,β-disubstituted to α,α-disubstituted β-lactone regioisomers, suggesting two possible reaction pathways. Epoxide ring-opening has traditionally been thought to occur via an S_{N}2 mechanism, wherein Co(CO)₄⁻ attacks the least-hindered oxirane carbon to give the steric product (Scheme 2, pathway A). However, an S_{N}1-type mechanism could also occur to produce the contrasteric product (FIG. 2, pathway B); the disubstituted position allows formation of a stable tertiary carbocation or partial positive charge that enables attack at the more sterically congested site.

The aldol reaction is one of the most widely used methods for carbon-carbon bond formation, both in nature and modern organic synthesis. While the stereoselective addition of an enolized carbonyl compound to an aldehyde has been well-established, addition to alkyl ketones has proven particularly challenging. This gap in the field is due to the inherent low reactivity and difficult enantiofacial discrimination of ketones, as well as rapid retro-reactions of the products. In 2001, Alper and co-workers also observed the generation of two β-lactone regioisomers from propylene oxide and CO when using a Co₂(CO)₈/BF₃.OEt₂ catalyst.

There is an unmet need for a method to produce β-lactones and ketone aldol products with high regioselectivity.

### SUMMARY OF THE DISCLOSURE

The present disclosure provides methods of carbonylating cyclic substrates to produce disubstituted lactones. These lactones may be transformed into ketone aldol products.

In an aspect, the methods of the present disclosure comprise a catalytic carbonylation and, optionally, a ring-opening reaction. In a method of the present disclosure, a cyclic substrate is reacted with CO in the presence of a catalyst to form a carbonylated cyclic product. A ring-opening reaction may be carried out on carbonylated cyclic products to form ketone aldol products. Methods of the present disclosure may be used to synthesize carbonylated cyclic products with high regioselectivity and the ketone aldol products may retain the stereochemistry and enantiomeric ratio of the carbonylated cyclic products.

For example, the present disclosure provides methods of producing carbonylated cyclic products. A method of the present disclosure may comprise providing a reaction mixture comprising providing a reaction mixture comprising a 2,2-disubstituted epoxide, a solvent or, and a catalyst (e.g., a [LA⁺][Co(CO)₄⁻] catalyst) in a vessel (e.g., a sealed vessel); and contacting (e.g., pressurizing) the reaction mixture with carbon monoxide. The temperature of the reaction mixture may be varied or held constant. For example, the temperature of the reaction mixture may be held for a selected time (e.g., 1 minute (m or min) to 96 hours (h or hr), including every second value and range therebetween) and/or temperature (e.g., −50 to 120° C., including every 0.1° C. value and range therebetween, such as, for example, 18 to 25° C. (e.g., 24° C.)), wherein the carbonylated cyclic product is produced. In various examples, the catalyst is formed in situ when providing a reaction mixture. A method of the present disclosure produces a β,β-disubstituted carbonyl product (e.g., β,β-disubstituted lactone). Methods of the present disclosure may be used to synthesize β,β-disubstituted lactones with high regioselectivity.

β,β-disubstituted carbonyl products (e.g., β,β-disubstituted lactones) may be opened by the methods described herein to form p-hydroxy esters. If the resulting lactone is purified before ring-opening, the overall two-step yield of the ring-opened product may improve. In various examples, β,β-disubstituted carbonyl products (e.g., β,β-disubstituted lactones) are contacted (e.g., reacted) with a base to form p-hydroxy esters.

### BRIEF DESCRIPTION OF DRAWINGS

For a fuller understanding of the nature and objects of the disclosure, reference should be made to the following detailed description taken in conjunction with the accompanying figures.

FIG. 1 depicts (A) the Mukaiyama Aldol Reaction and (B) an alternative epoxide carbonylation/ring-opening route.

FIG. 2 depicts steric and contrasteric ring-opening for the carbonylation of 2,2-disubstituted epoxides.

FIG. 3 depicts a proposed mechanism for the carbonylation of 2,2-disubstituted epoxides.

FIG. 4 depicts stereochemistry of carbonylation and ring-opening steps.

FIG. 5 depicts [Lewis Acid(THF)₂]⁺[Co(CO)₄]⁻ catalysts screened for isobutylene oxide carbonylation.

FIG. 6 depicts scope of alkyl 2,2-disubstituted epoxides.

FIG. 7 depicts previous carbonylation of isobutylene oxide suffering from byproduct formation and low regioselectivities.

FIG. 8 depicts [Lactone]ₜ dependence on [IBO]₀ (tᵣₓₙ=2 h for THF and tᵣₓₙ=0.25 h for ⁱPr₂O) in different solvents using catalyst 1f. IBO=Isobutylene oxide.

FIG. 9 depicts the rate dependence of P_{CO}.

FIG. 10 depicts the rate dependence on [1f] in ⁱPr₂O.

FIG. 11 depicts chiral HPLC analysis of racemic 2m.

FIG. 12 depicts chiral HPLC analysis of (R)-2m.

FIG. 13 depicts chiral HPLC analysis of racemic 3m.

FIG. 14 depicts chiral HPLC analysis of (R)-3m.

FIG. 15 depicts chiral HPLC analysis of racemic 6m.

FIG. 16 depicts chiral HPLC analysis of (R)-6m.

FIG. 17 depicts carbonylation results using a bislactone substrate.

FIG. 18 depicts A. shows an NMR spectrum of products depicted in FIG. 18. B. depicts the spectrum of A. zoomed in.

FIG. 19 depicts chiral GC analysis of racemic 2q.

FIG. 20 depicts chiral GC analysis of (R)-2q.

FIG. 21 depicts chiral GC analysis of racemic 3q.

FIG. 22 depicts chiral GC analysis of (R)-3q.

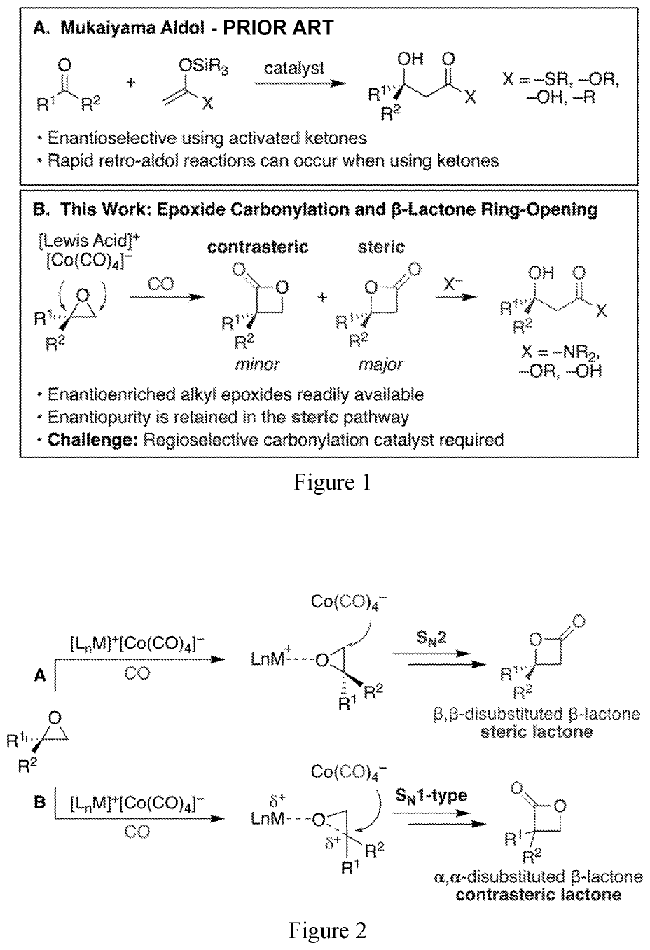

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

All ranges provided herein include all values that fall within the ranges to the hundredth decimal place, unless indicated otherwise.

The present disclosure provides methods of carbonylating cyclic substrates to produce disubstituted lactones with high regioselectivity. These lactones may be transformed into ketone aldol products.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). Illustrative examples of groups include:

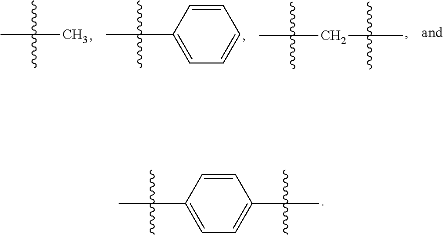

As used herein, unless otherwise indicated, the term “aliphatic” refers to branched or unbranched hydrocarbon groups that, optionally, contain one or more degrees of unsaturation. Degrees of unsaturation can arise from, but are not limited to, aryl groups and cyclic aliphatic groups. For example, the aliphatic groups/moieties are a C₁ to C₄₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The aliphatic group can be unsubstituted or substituted with one or more substituent. Examples of substituents include, but are not limited to, substituents such as, for example, halogens (—F, —Cl, —Br, and —I), aliphatic groups (e.g., alkyl groups and the like), halogenated aliphatic groups (e.g., trifluoromethyl group), aryl groups, halogenated aryl groups, substituted amine groups, carboxylic acids groups, protected alcohol groups, ether groups, ester groups, thioether groups, thioester groups, substituted carbamate groups, substituted amide groups, alkenes with a long alkyl chain between connecting it to the epoxide, and the like, and combinations thereof.

As used herein, unless otherwise indicated, the term “alkyl” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₄₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The alkyl group may be unsubstituted or substituted with one or more substituents. Examples of substituents include, but are not limited to, substituents such as, for example, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

In an aspect, the methods of the present disclosure comprise a catalytic carbonylation and, optionally, a ring-opening reaction. In a method of the present disclosure, a cyclic substrate is reacted with CO in the presence of a catalyst to form a carbonylated cyclic product. A ring-opening reaction may be carried out on carbonylated cyclic products to form ketone aldol products. Methods of the present disclosure may be used to synthesize carbonylated cyclic products with high regioselectivity and the ketone aldol products may retain the stereochemistry and enantiomeric ratio of the carbonylated cyclic products.

For example, the present disclosure provides methods of producing carbonylated cyclic products. A method of the present disclosure may comprise providing a reaction mixture comprising providing a reaction mixture comprising a 2,2-disubstituted epoxide, a solvent or, and a catalyst (e.g., a [LA⁺][Co(CO)₄⁻] catalyst) in a vessel (e.g., a sealed vessel); and contacting (e.g., pressurizing) the reaction mixture with carbon monoxide. The temperature of the reaction mixture may be varied or held constant. For example, the temperature of the reaction mixture may be held for a selected time (e.g., 1 minute (m or min) to 96 hours (h or hr), including every second value and range therebetween) and/or temperature (e.g., −50 to 120° C., including every 0.1° C. value and range therebetween, such as, for example, 18 to 25° C. (e.g., 24° C.)), wherein the carbonylated cyclic product is produced. In various examples, the catalyst is formed in situ when providing a reaction mixture. A method of the present disclosure produces a β,β-disubstituted carbonyl product (e.g., β,β-disubstituted lactone). Methods of the present disclosure may be used to synthesize β,β-disubstituted lactones with high regioselectivity.

Carbonylated cyclic products (e.g., β,β-disubstituted carbonyl compounds, such as, for example, β,β-disubstituted lactones) may be opened by the methods described herein to form β-hydroxy esters. If the resulting lactone is purified before ring-opening, the overall two-step yield of the ring-opened product may improve. In various examples, β,β-disubstituted carbonyl products (e.g., β,β-disubstituted lactones) are contacted (e.g., reacted) with a base to form β-hydroxy esters.

Ketone aldol products (e.g., β-hydroxy esters) may be produced by a method of the present disclosure via a “one pot” procedure. Following formation of a β,β-disubstituted carbonyl product (e.g., a β,β-disubstituted lactone) using the methods described herein, carbon monoxide may be vented from the reaction mixture and base (e.g., sodium methoxide in methanol) may be added directly to the crude β,β-disubstituted carbonyl product (e.g., a β,β-disubstituted lactone) mixture. The resulting ketone aldol product (e.g., p-hydroxy ester) may be isolated by methods known in the art. Such ketone aldol products may have high regioselectivity (e.g., 90:10, 91:9, 92:8, 93:7, 94:6, 95:5, 96:4, 97:3, 98:2, 99:1, or >99:1). The ketone aldol product may be described as having a high enantiopurity (e.g., greater than 90%, greater than 95%, or greater than 99%). In various examples, the ketone aldol product retains the stereochemistry and enantiomeric ratio of the β,β-disubstituted carbonyl product (e.g., a β,β-disubstituted lactone).

Various bases are may be used to produce β-hydroxy esters from lactones. Non-limiting examples of bases are alkoxide bases and amine bases. Non-limiting examples of alkoxide bases include sodium methoxide, sodium ethoxide, sodium n-propoxide, sodium isopropoxide, sodium tert-butoxide, and the like, and combinations thereof. Non-limiting examples of amine bases include primary and secondary amines. Examples of amines include, but are not limited to, primary alkyl amines (e.g., methylamine, ethylamine, propylamine, isopropylamine, isopropylamine, butylamine, tert-butylamine, amylamine, hexylamine, cyclohexylamine, phenethylamine, and the like), secondary alkyl amines (e.g., dimethylamine, ethylmethylamine, diethylamine, dipropylamine, and the like), cyclic amines (e.g., pyrrolidine, piperidine, and the like), and the like.

The method may be carried out by reacting (e.g., pressurizing) a reaction mixture comprising an epoxide(s), carbon monoxide, a solvent, and a carbonylation catalyst (e.g., a [LA⁺][Co(CO)₄⁻] catalyst) to form a product (e.g., lactone(s) and, optionally, one or more minor product(s)).

The carbonylation reaction to form the lactone can be allowed to proceed to a desired extent prior to release of any residual carbon monoxide. For example, the carbonylation reaction can be monitored using in situ IR spectroscopy to determine the extent of epoxide reaction and lactone formation. In an example, the carbonylation can be allowed to proceed until there is no detectible epoxide (for example, by IR spectroscopy) prior to release of any residual carbon monoxide and addition of the polymerization catalyst.

Various solvents may be used. Examples of suitable solvents include, but are not limited to, tetrahydrofuran, 2-methyltetrahydrofuran, 2,5-dimethyltetrahydrofuran, alkyl ethers (e.g., diethylether, diisopropyl ether, and the like), benzene, toluene, 1,4-dioxane, or a mixture thereof. It is desirable that the solvent be a non-chlorinated, aprotic solvent. Without intending to be bound by any particular theory, it is considered that weakly coordinating, bulky ethers tend to increase the overall rate of reaction. Additional examples of solvents include, but are not limited to, alkyl acetates, such as, for example, ethyl acetates and the like.

Various cyclic substrates may be used. A cyclic substrate may be a 2,2-disubstituted epoxide. Examples of 2,2-disubstituted epoxides include, but are not limited to, isobutylene oxide, as well as epoxides having the following structure:

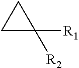

where R₁═R₂=the same alkyl group, epoxides where R₁=one alkyl group and R₂=another alkyl group, R₁=Me and R₂=an alkyl group. In various examples, R₁ and R₂, are joined to form an optionally cyclic alkyl ring. The term “cyclic alkyl ring,” refers to monocyclic and polycyclic ring systems having a total of five to 20 atoms in the ring, where, for polycyclic ring systems, each ring in the system contains three to twelve ring members. Non-limiting examples of 2,2-disubstituted epoxides include:

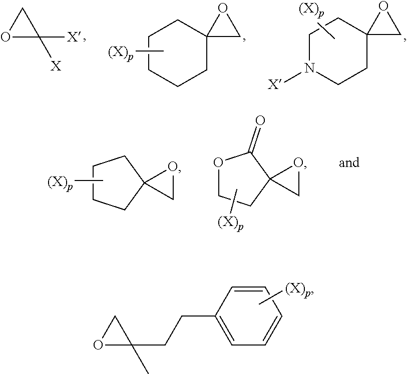

where X and X′ at each occurrence is independently chosen from H, alkoxy groups, alkyl groups (e.g., branching or linear substituted or unsubstituted alkyl groups), aryl groups, halides, amino groups, alkenyl groups, alkynyl groups, acyl groups, nitro groups, nitrile groups, hydroxyl groups, protected hydroxyl groups, ether groups, ester groups, thioester groups, polymeric groups, and thioether groups, and p is 0-10. Additional examples include, but are not limited to,

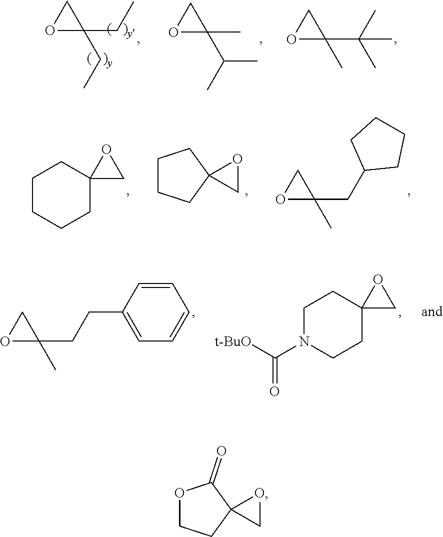

where y and y′ are independently 0-40, including every integer value and range therebetween (e.g., 1, 2, 3, 4, 5, 6, 7, 8, 9, 10, 11, 12, 13, 14, 15, 20, 30, 40). In various examples, a 2,2-disubstituted epoxide does not have the following structure:

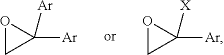

where Ar is an aryl group and X is chosen from H, alkoxy, alkyl, aryl groups, halides, amino groups, alkenyl groups, alkynyl groups, acyl groups, nitro groups, nitrile groups, hydroxyl groups, protected hydroxyl groups, ether groups, ester groups, thioester groups, and thioether groups, and the like, and combinations thereof.

The epoxides may be present as mixtures of stereoisomers, where the mixture is, for example, enriched in one stereoisomer relative the complementary stereoisomer (also referred to as an optically enriched form of the epoxides) and/or racemic mixtures of epoxides. For example, the epoxides can be present as a mixture of stereoisomers having greater than 90, 95.99, 99.5, 99.9% purity of one stereoisomer. In another example, optically pure (i.e., no detectible complementary stereoisomer present) epoxide is used. Optical purity can be determined by, for example, chiral gas chromatography or chiral high performance liquid chromatography. Use of optically pure epoxides or epoxides present a mixture enriched, e.g., greater than 90% enrichment, in a stereoisomer in the methods described herein can result in formation of optically enriched O-lactones and ring-opened aldol products (e.g., a ketone aldol product).

In various examples, enantiopurity of a starting substrate (e.g., a cyclic substrate such as, for example, an epoxide) is maintained throughout carbonylation and ketone aldol formation (e.g., methanolysis) to result in a product (e.g., a lactone or a ketone aldol product) with retention of stereochemistry.

Methods of the present disclosure may have a desirable regioselectivity. For example, carbonylation products and ketone aldol products produced by methods of the present can be formed with a regioselectivity of 90:10, 91:9, 92:8, 93:7, 94:6, 95:5, 96:4, 97:3, 98:2, 99:1, or >99:1.

Methods of the present disclosure may have desirable turnover frequencies ((mol starting material consumed)/[(mol catalyst)·(tᵣₓₙ in hours)]). For example, methods of the present disclosure have a turnover frequency of 1-10,000, including all integer values and ranges therebetween (e.g., 1-1000, 1-500, 1-100, 1-50, 1-20).

Methods of the present disclosure may have desirable turnover numbers. For example, methods of the present disclosure have a turnover number of 1-10,000, including all integer values and ranges therebetween (e.g., 1-1000, 1-500, 1-100, 1-50, 1-20).

Various carbonylation catalysts can be used. A carbonylation catalyst may comprise a Lewis acid cation and a cobalt carbonyl anion. It is expected that a various range of Lewis acids may be used to produce various lactone products, however, regioselectivity may suffer using highly Lewis acidic catalysts. Without intending to be bound by any particular theory, it is considered the less electron-donating the catalyst substituents, the more likely steric selectivity will be achieved. Mixtures of carbonylation catalysts may be used

A carbonylation catalyst may be a [LA⁺][Co(CO)₄⁻] catalyst, where [LA⁺] is a Lewis acid capable of coordinating an ether, such as, for an example, alkyl ethers (e.g., diethyl ether, diisopropyl ether, ethylmethyl ether, t-butylmethyl ether, and the like), cyclic ethers (e.g., THF, furan, oxane, and the like), and the like. Without intending to be bound by any particular theory, it is considered that a Lewis acid that coordinates an ether will also bind a cyclic substrate (e.g., an epoxide). [LA⁺] may be a porphyrin-based Lewis acid cation or a salph/salen-based Lewis acid cation. In various examples, a carbonylation catalyst comprises a porphyrin group or a salph group or a salen group.

Coordinating an ether to a carbonylation catalyst may be determined using x-ray crystallography. For example, a catalyst is crystallized out of a solution comprising the catalyst and an ether and x-ray crystallography is performed on the crystallized catalyst. When, based on the crystal structure, the ether oxygen is within a coordinating distance from the metal of the Lewis acid (e.g., less than 3 Å), the ether is considered coordinated to the Lewis acid.

Carbonylation catalysts of the present disclosure may be preformed or made in situ. For example, a metal salt catalyst (e.g., MCI, where M is a metal, such as, for example, aluminum) is added to added to the reaction mixture with a Co(CO)₄ salt (e.g., NaCo(CO)₄). In a more specific example, an Al—Cl catalyst is mixed in a solvent with NaCo(CO)₄ and the active complex is formed as depicted in the following schemes:

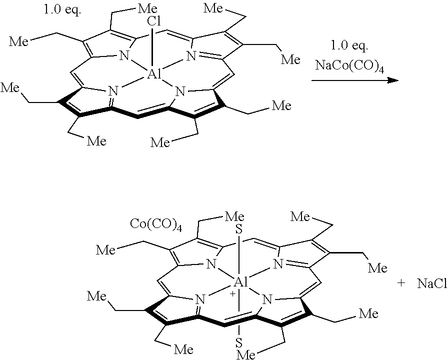

A non-limiting example of in situ formation is depicted in the following scheme:

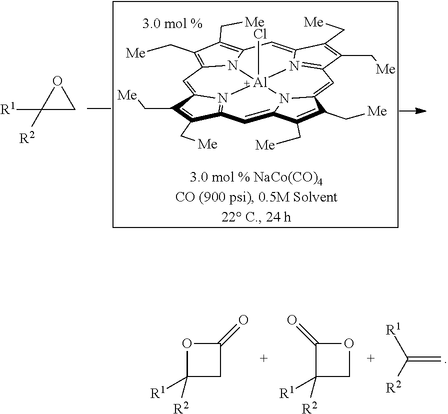

A porphyrin-based Lewis acid catalyst may have the following structure:

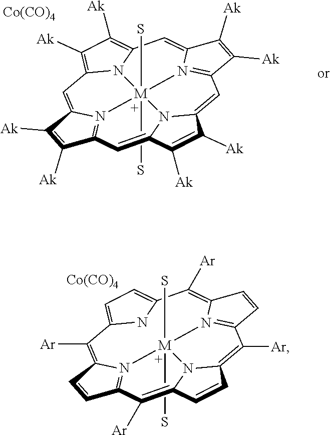

where S is solvent (e.g., THF), M is a metal (e.g., aluminum or chromium), Ak is a substituted linear or branched C₁ to C₁₀ alkyl group or unsubstituted linear or branched C₁ to C₁₀ alkyl group (e.g., methyl, ethyl, propyl, isopropyl, butyl, t-butyl, and the like, and combinations thereof), where each Ak may be the same or different, and Ar is a substituted or unsubstituted aryl group, where each Ar may be the same or different. The aryl groups may be substituted with halogen groups, alkoxy groups (e.g., methoxy groups), or various substituted linear or branched C₁ to C₁₀ alkyl groups or unsubstituted linear or branched C₁ to C₁₀ alkyl groups. In various examples, Ar is chosen from phenyl, p-ClC₆H₄, p-FC₆H₄, p-OMeC₆H₄, and 2,4,6-Me₃C₆H₂. In various examples Ak is ethyl. Various metals may be used, such as, for example, aluminum, chromium, and the like. In various examples, the metal is aluminum.

A salph/salen-based Lewis acid catalyst has the following structure:

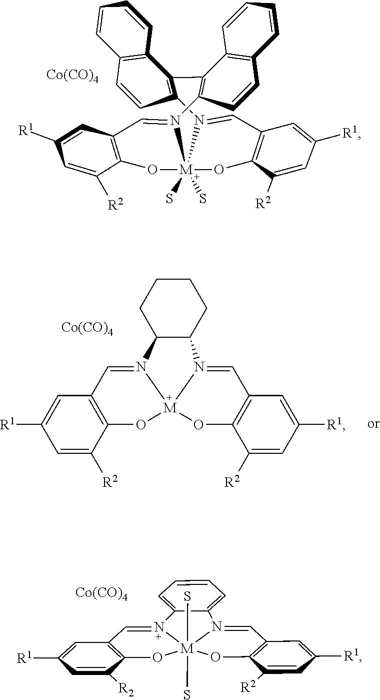

where S is solvent (e.g., THF), M is a metal (e.g., aluminum or chromium), and R¹ and R² are independently chosen from H, alkyl groups (e.g., methyl, ethyl, propyl, isopropyl, butyl, t-butyl and the like, and combinations thereof), halogen groups, and alkoxide groups. R¹ and R² may be the same or different. Various metals may be used, such as, for example, aluminum, chromium, and the like. In various examples, the metal is aluminum.

Examples of suitable catalysts include, but are not limited, to N,N′-Bis(3,5-di-tert-butylsalicylidene)-1,2-phenylenediaminoaluminum cobalt tetracarbonyl [(salen)Al(THF)₂]⁺[Co(CO)₄]⁻; Bis(tetrahydrofuran)-meso-tetraphenylporphyrinato aluminum tetracarbonyl cobaltate, [(TPP)Al(THF)₂]⁺[Co(CO)₄]⁻; Bis(tetrahydrofuran)-meso-tetra(4-chlorophenyl)porphyrinato aluminum tetracarbonyl cobaltate, [(4-ClTPP)Al(THF)₂]⁺[Co(CO)₄]⁻; Bis(tetrahydrofuran)-meso-tetra(4-methoxyphenyl)porphyrinato aluminum tetracarbonyl cobaltate, [(4-OMeTPP)Al(THF)₂]⁺[Co(CO)₄]⁻; Bis(tetrahydrofuran)-meso-tetra(2,4,6-trimethylphenyl)porphyrinato aluminum tetracarbonyl cobaltate, [(2,4,6-trimethylTPP)Al(THF)₂]⁺[Co(CO)₄]⁻; Bis(tetrahydrofuran)-octaethylporphyrinato aluminum tetracarbonyl cobaltate, [(OEP)Al(THF)₂]⁺[Co(CO)₄]⁻.

The carbonylation catalyst (e.g., a [LA⁺][Co(CO)₄⁻] catalyst) can be present in the reaction mixture at a concentration (e.g., catalyst loading) of 10 mol % or less relative to the cyclic substrate (e.g., 2,2-disubstituted epoxide). For example, the carbonylation catalyst has a concentration of 0.0001 to 10 mol %, including all 0.0001 mol % values and ranges therebetween (e.g., 0.01 to 10 mol %, 0.1 to 10 mol %) (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 5, 6, 7, 8, 9, or 10 mol %). In various examples, the concentration of the catalyst is 0.5-10 mol %. In various other examples, the concentration of the catalyst is 0.5-5 mol %. In various other examples, the concentration of the catalyst is 0.01-5 mol %. In various other examples, the concentration of the catalyst is 0.1 mM to 0.2 M, including all values to the 0.1 mM and ranges therebetween (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 5, 6, 7, 8, 9, 10, 20, 30, 40, 50, 60, 70, 80, 90, 100, 110, 120, 130, 140, 150, 160, 170, 180, 190, or 200 mM).

In various examples, reaction times and conditions (e.g., temperature and carbon monoxide pressure) for the method can be varied to achieve the desired result. For example, method times of approximately 24 hours are utilized. For example, the temperature is limited to 22° C. in an effort to, without intending to be bound by any particular theory, avoid the generation of undesired byproducts. In various examples, a desired temperature is −50-120° C. (e.g., 15-65° C.), including all 0.1° C. values and ranges therebetween (e.g., 20, 21, 22, 23, 24, 25, 26, 27, 28, 29, 30, 35, 40, 45, 50, 55, 60, or 65° C.). The reaction mixture may be held at a constant temperature or the temperature of the reaction mixture may be modulated. For example, the reaction mixture is heated or cooled to a desired temperature or temperatures before, after, and/or during pressurization. The reaction mixture may be held at a desired temperature or temperatures for a period of time (e.g., ranging from 1 second (s) to over 24 hours (h or hrs) or over 48 h or over 72 h or over 96 h). Alternatively, the temperature of the reaction mixture may be maintained at a constant temperature for the entirety of the method. For example, the temperature of the reaction mixture is −50 to 120° C., including every 0.1° C. value and range therebetween, such as, for example, 18 to 25° C. (e.g., 24° C.), for any period of time during the method.

The carbon monoxide may be present as a static atmosphere (e.g., a sealed reaction vessel) or as a stream (e.g., a flow-type reactor). The carbon monoxide pressure may be 1 atm to 100 atm, including all 0.1 atm values and ranges therebetween. The pressure of the method may also be described in psi. For example, the carbon monoxide pressure may be psi to 1500 psi, including every integer psi value and range therebetween (e.g., 100 psi to 1500 psi). In various examples, the reactions were run at 22° C. with 61.24 atm (900 psi) carbon monoxide.

Methods of the present disclosure may be used to synthesize β,β-disubstituted lactones with high regioselectivity (e.g., 90:10, 91:9, 92:8, 93:7, 94:6, 95:5, 96:4, 97:3, 98:2, 99:1, or >99:1). The β,β-disubstituted lactones may be converted (e.g., ring opened, such as, for example, through nucleophilic addition) to form ketone aldol products (e.g., β-hydroxy esters) by addition of a base (e.g., sodium methoxide in methanol). The β,β-disubstituted lactones may be optionally purified before conversion to a ketone aldol product.

An amount of one or more minor product(s) may be produced during the formation of β,β-disubstituted lactones by a method of the present disclosure. The one or more minor product(s) may include α,α-disubstituted lactones and alkenes. In various examples, 10% or less, 9% or less, 8% or less, 7% or less, 6% or less, 5% or less, 4% or less, 3% or less, 2% or less, 1% or less, or less than 1% of the one or more minor product(s) is formed by a method of the present disclosure.

The steps of the methods described in the various examples disclosed herein are sufficient to produce a compound of the present disclosure. Thus, in an embodiment, a method consists essentially of a combination of the steps of the methods disclosed herein. In another embodiment, a method consists of such steps.

The following Statements describe various non-limiting examples of methods of the present disclosure:

Statement 1. A method of carbonylating a cyclic substrate comprising: i) providing a reaction mixture comprising a cyclic substrate, a solvent, carbon monoxide, and a catalyst (e.g., a [LA⁺][Co(CO)₄⁻] catalyst, where [LA⁺] is a Lewis acid capable of coordinating to a cyclic substrate.); ii) pressurizing the reaction mixture, wherein a carbonylated cyclic substrate (e.g., a carbonylated cyclic product) is formed. The carbonylated cyclic product (e.g., β,β-disubstituted lactone) may be isolated using methods known in the art. The reaction mixture may be held at a constant temperature for all of or a portion of the course of the method or the temperature is modulated during the course of the method. The [LA⁺][Co(CO)₄⁻] catalyst may have a Lewis acid cation that is porphyrin-based or salph/salen-based.
Statement 2. A method according to Statement 1, where the porphyrin-based Lewis acid catalyst has the following structure:

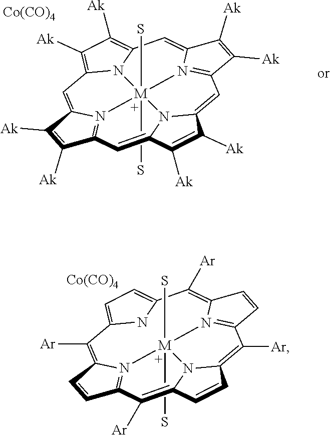

where S is solvent, M is a metal chosen from aluminum and chromium, Ak is a substituted linear or branched C₁ to C₁₀ alkyl group or unsubstituted linear or branched C₁ to C₁₀ alkyl group (e.g., each Ak is an ethyl group), and Ar is a substituted or unsubstituted aryl group.
Statement 3. A method according to Statement 2, wherein Ar is chosen from phenyl, p-ClC₆H₄, p-FC₆H₄, p-OMeC₆H₄, and 2,4,6-Me₃C₆H₂.
Statement 4. A method according to any one of the previous Statements, where the porphyrin-based Lewis acid catalyst is chosen from Bis(tetrahydrofuran)-meso-tetraphenylporphyrinato aluminum, [(TPP)Al(THF)₂]⁺, Bis(tetrahydrofuran)-meso-tetra(4-chlorophenyl)porphyrinato aluminum, [(4-ClTPP)Al(THF)₂]⁺, Bis(tetrahydrofuran)-meso-tetra(4-methoxyphenyl)porphyrinato aluminum, [(4-OMeTPP)Al(THF)₂]⁺, Bis(tetrahydrofuran)-meso-tetra(2,4,6-trimethylphenyl)porphyrinato aluminum, [(2,4,6-trimethylTPP)Al(THF)₂]⁺, and Bis(tetrahydrofuran)-octaethylporphyrinato aluminum, [(OEP)Al(THF)₂]⁺.
Statement 5. A method according to Statement 1, wherein the salph/salen-based Lewis acid catalyst has the following structure:

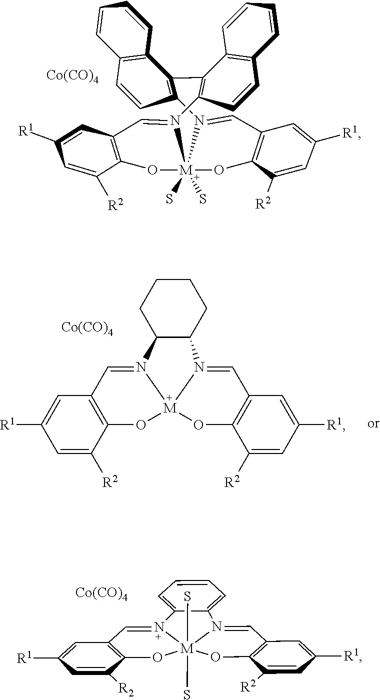

where S is a solvent, M is a metal chosen from aluminum and chromium, and R¹ and R² are independently chosen from H, alkyl groups (e.g., methyl, ethyl, propyl, isopropyl, butyl, t-butyl and the like), halogen groups, and alkoxide groups (e.g., methoxy, ethoxy, propoxy, and the like).
Statement 6. The method of claim 4, wherein the salph/salen-based Lewis acid catalyst is N,N′-Bis(3,5-di-tert-butylsalicylidene)-1,2-phenylenediaminoaluminum, [(salen)Al(THF)₂]⁺.
Statement 7. A method according to any one of the preceding Statements, where the carbonylated cyclic product has a regioselectivity of at least 90:10.
Statement 8. A method according to any one of the preceding Statements, where a mixture of products is formed and the mixture of products comprises a major product and one or more minor product(s), and the major product is the β,β-disubstituted lactone.
Statement 9. A method according to Statement 8, where the one or more minor product(s) comprise an α,α-disubstituted lactone.
Statement 10. A method according to Statement 8 or Statement 9, where 10% or less of the mixture of products comprises the one or more minor product(s).
Statement 11. A method according to any one of the preceding Statements, further comprising contacting the β,β-disubstituted lactone with a base and, optionally, isolating a (3-hydroxy ester.
Statement 12. A method according to Statement 11, where the base is an alkoxide base.
Statement 13. A method according to Statement 12, where the alkoxide base is chosen from sodium methoxide, sodium ethoxide, sodium n-propoxide, sodium isopropoxide, and sodium tert-butoxide.
Statement 14. A method according to any one of the preceding Statements, where the pressurizing is performed via charging with carbon monoxide.
Statement 15. A method according to Statement 14, where the pressurizing is continuously charging with a stream of carbon monoxide or charging to a static environment of carbon monoxide.
Statement 16. A method according to any one of the preceding Statements, where the cyclic substrate is a 2,2,-disubstituted epoxide.
Statement 17. A method according to Statement 16, where the 2,2-disubstituted epoxide is chosen from:

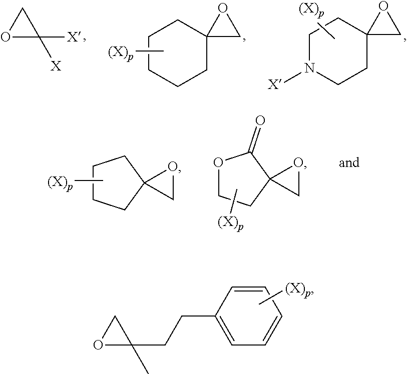

where X and X′ at each occurrence is independently chosen from H, alkoxy, alkyl, aryl groups, halides, amino groups, alkenyl groups, alkynyl groups, acyl groups, nitro groups, nitrile groups, hydroxyl groups, protected hydroxyl groups, ether groups, ester groups, thioester groups, and thioether groups, and p is 0-10.
Statement 18. A method according to Statement 17, where the 2,2-disubstituted epoxide is chosen from:

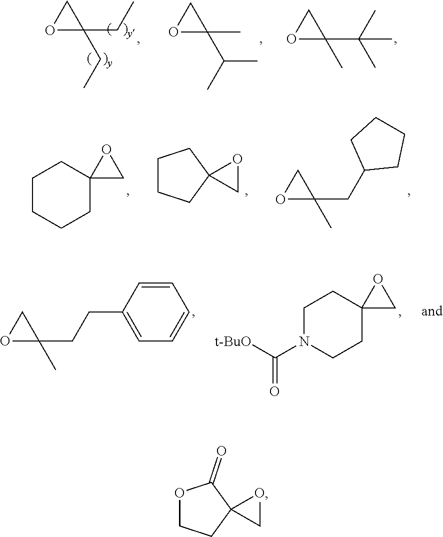

wherein y and y′ are independently 0-40, including every integer value and range therebetween (e.g., 1, 2, 3, 4, 5, 6, 7, 8, 9, 10, 11, 12, 13, 14, 15, 20, 30, 40).
Statement 19. A method according to any one of the preceding Statements, where the solvent is chosen from tetrahydrofuran, 2-methyltetrahydrofuran, 2,5-dimethyltetrahydrofuran, diethylether, diisopropyl ether, benzene, toluene, 1,4-dioxane, and combinations thereof.
Statement 20. A method according to any one of the preceding Statements, where the carbon monoxide pressure is 1 atm to 100 atm.
Statement 21. A method according to any one of the preceding Statements, where the catalyst is at a concentration of 0.0001 to 10 mol % relative to the cyclic substrate.
Statement 22. A method according to any one of the preceding Statements, wherein the reaction mixture is at a temperature of −50 to 120° C. The temperature of the reaction mixture may be constant or varied during the method.

The following example is presented to illustrate the present disclosure. It is not intended to be limiting in any matter.

### Example

The following example describes a method of the present disclosure.

Reported is the regioselective carbonylation of 2,2-disubstituted epoxides to β,β-disubstituted β-lactones. A wide range of epoxides can be carbonylated to β-lactones, which are subsequently ring-opened to produce ketone-based aldol adducts, providing an alternative to the Mukaiyama aldol reaction. An enantiopure epoxide was demonstrated to undergo the carbonylation/ring-opening process with retention of stereochemistry to form an enantiopure β-hydroxy ester.

Catalyst optimization. A first generation carbonylation catalyst [(salph)Al(THF)₂]⁺[Co(CO)₄]⁻ (1a) displayed low conversion and moderate regioselectivity for isobutylene oxide carbonylation at 22° C. (Table 1, entry 1). Catalyst variations with modified ortho-substituents (1g, Table 1, entry 7) and alternative backbones (1h and 1i, Table 1, entries 8 and 9) displayed attenuated activity and no improvement to regioselectivity. Although conversions improved using complexes 1b, 1c and 1j (Table 1, entries 2, 3 and 10), regioselectivity remained modest (93:7). The Lewis acidity was reduced to facilitate steric attack; however, introducing electron-donatingp-OMe groups (1d, Table 1, entry 4) or mesityl rings (1e, Table 1, entry 5) decreased conversion with no change in regioselectivity. Next, we screened an alkyl-substituted porphyrin (1f, Table 1, entry 6) to shift electron density onto the metal center. This octaethylporphyrin catalyst exhibited the highest regioselectivity (96:4) while maintaining good activity.

Mechanistic investigations. In light of previous reports studying the effect of solvent donicity on carbonylation reactions, a screen was performed to further improve reaction rate and regioselectivity (Table 2). Similar to these reports, strongly donating acetonitrile (MeCN) coordinated to the catalyst, inhibiting epoxide binding and ultimately preventing reactivity (Table 2, entry 1). Reactions conducted in THF and 1,4-dioxane, common coordinating solvents, proceeded with a turnover frequency (TOF) of 10 h⁻¹ and regioselectivities of 96:4 and 95:5, respectively (Table 2, entries 2 and 3). However, less donating ethers such as 2,5-dimethyltetrahydrofuran (DMTHF), diethyl ether (Et₂O), and diisopropylether (ⁱPr₂O) (Table 2, entries 4-6) facilitated faster reaction rates (TOF=14-17 h⁻¹) and increased regioselectivities to >99:1. Non-coordinating toluene and hexanes (Table 2, entries 7 and 8) did not shut down reactivity, but neither were competitive with ethereal solvents. This decrease in activity indicates that a minimal amount of donicity is required for reaction turnover with weakly donating, ethereal solvents maximizing both TOF and regioselectivity.

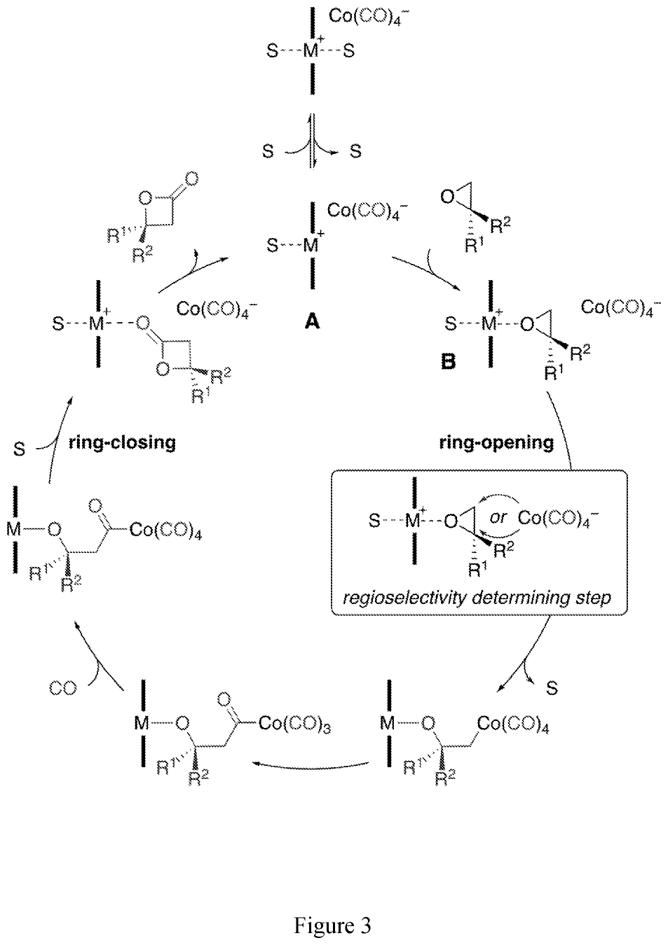

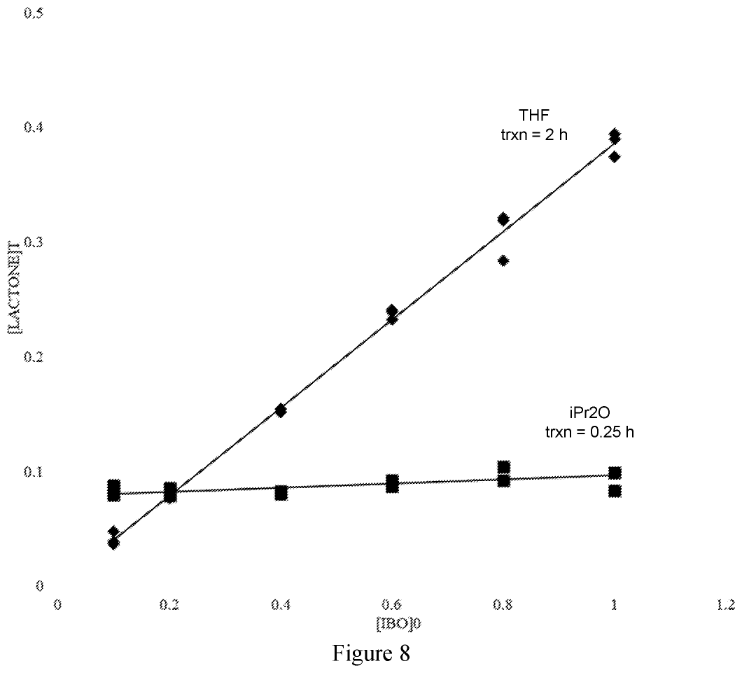

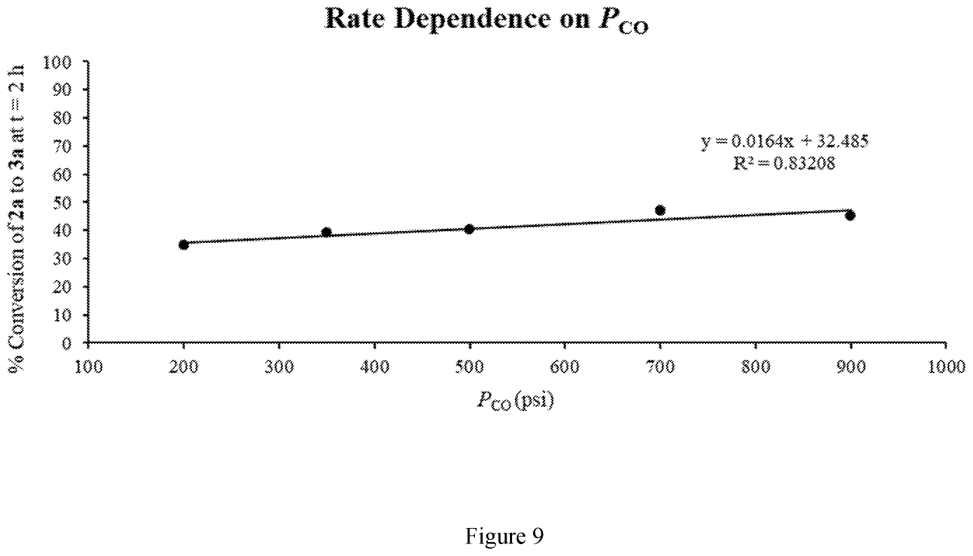

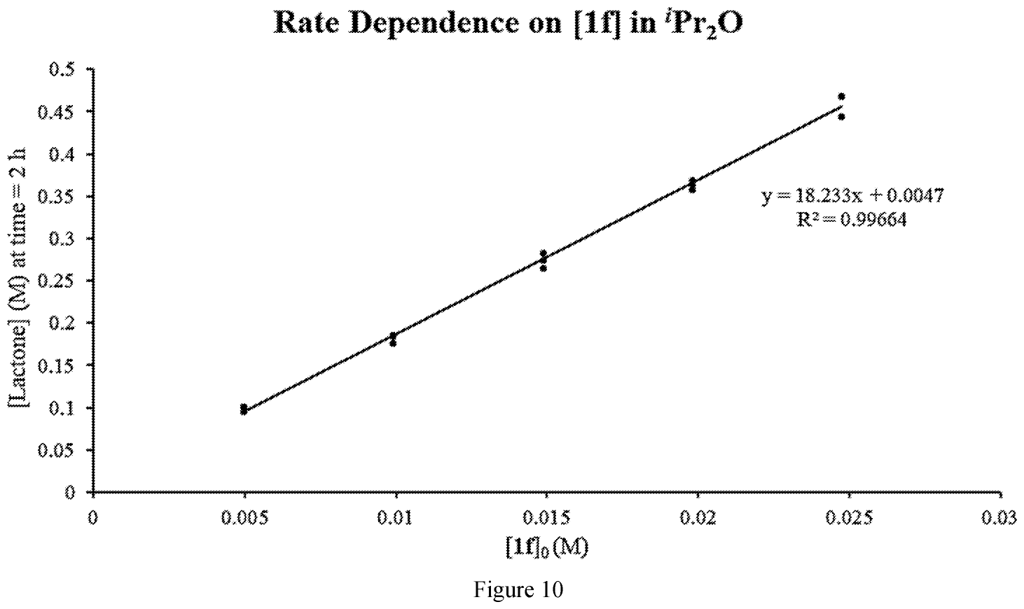

However, in the case of 2,2-disubstituted epoxides, weakly donating solvents accelerated the reaction rate, prompting new mechanistic studies. To better understand this different trend, the reaction order in isobutylene oxide in both THF and ⁱPr₂O was determined (FIG. 8). Unlike monosubstituted epoxides, which demonstrate a zero-order trend in epoxide using THF, isobutylene oxide showed first-order behavior (FIG. 8). It is believed mechanistic difference is due to the addition of a second methyl group to the substituted carbon of the epoxide. This geminal dimethyl group likely facilitates ring closure via the Thorpe-Ingold effect, ultimately changing the turnover limiting step from ring-closing to ring-opening in THF (FIG. 3).

This hypothesis is supported by the zero-order dependence on initial epoxide concentration in ⁱPr₂O (FIG. 8). Because an axially bound solvent molecule dissociates during epoxide ring-opening, bulky and weakly coordinating solvents likely facilitate this step, implying the restoration of turnover limiting ring-closure. However, it cannot be ruled out a change in catalyst resting state between unbound (FIG. 3, part A) and bound epoxide (FIG. 3, part B) with an accelerated turnover limiting ring-opening step. A first-order dependence on 1f concentration was determined, which is consistent with this mechanism. Less coordinating solvents such as toluene and hexanes likely exhibit decreased rates because ring closure still requires the binding of a solvent molecule.

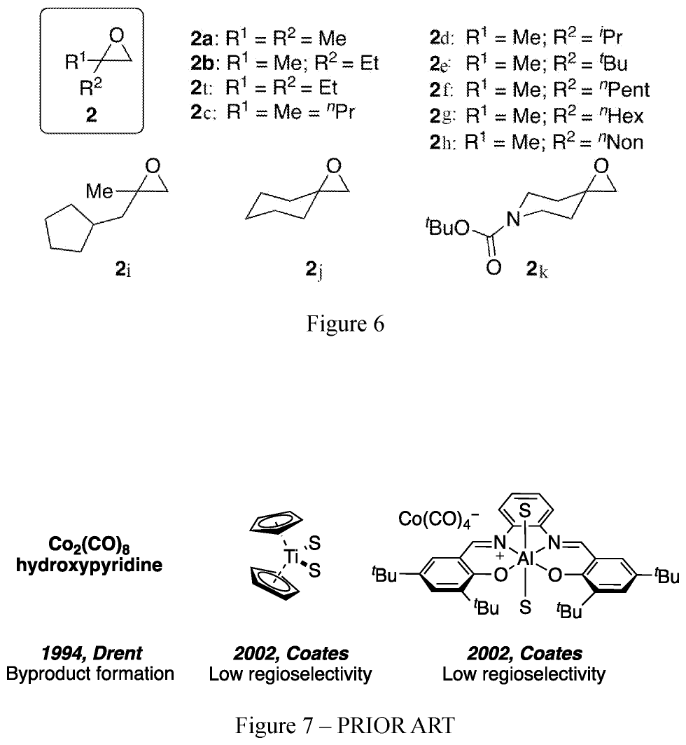

Carbonylation substrate scope and aldol product formation. Using these optimized conditions, alkyl 2,2-disubstituted epoxides (FIG. 6) were carbonylated to afford various β,β-disubstituted β-lactones with minimal side product formation (vide infra, 5, Table 3). Substrates with alkyl groups (Table 3, entries 1-8) were carbonylated in high yields and with >98:2 regioselectivity. Additional steric hindrance close to the epoxide substrate was also tolerated. Isopropyl and tert-butyl substitution (2d and 2e, Table 3, entries 4 and 5) produced exclusively steric lactone in 79 and 70% isolated yield, respectively. Although regioselectivity was enhanced with increased steric bulk, the carbonylation reaction using 2e was slow and required a higher catalyst loading to reach comparable conversion in the same amount of time. However, both regioselectivity and reaction rate remained high when the steric bulk was located one methylene unit farther from the quaternary center (2i, Table 3, entry 9). Spirocyclic epoxides, 2j and 2k, were also carbonylated successfully (entries 10 and 11). The ethyl, ethyl substituted derivative, 2t, yielded 94:6 regioselectivity.

Each reaction exhibits an alkenyl decarboxylation side product (5, Table 3), which has been observed in similar transformations, indicating a competing reaction pathway. Previous reports show evidence of a lactone [2+2] cycloreversion mechanism that proceeds by way of a zwitterionic intermediate featuring a carboxylate and tertiary carbocation (⁻O₂CCH₂CR₂⁺). β-Substitution of the steric lactone (3) stabilizes the carbocation intermediate and facilitates the proposed pathway, but the contrasteric product cannot undergo the cycloreversion as it would require the formation of an unstable primary carbocation. This side reaction is avoided by stopping the reactions immediately after, or just before, full conversion is reached.

Other 2,2-disubstituted epoxides did not require 1f to achieve high regioselectivities, so cheaper and more easily synthesized 1b was used instead (Table 4). Aryl-substituted epoxides 2l, 2m, and 2n (Table 4, entries 1-3) were carbonylated in moderate yield and excellent regioselectivity. A methyl-substituted analogue of epichlorohydrin (20, Table 4, entry 4) produced exclusively steric lactone in 84% isolated yield, and substrates with TBS-protected alcohols (2p and 2q, Table 4, entries 5 and 6) were also carbonylated efficiently. Methacrylate-derived substrates 2r and 2s (Table 4, entries 7 and 8) required higher temperatures and longer reaction times, but regioselectivity remained desirable.

The class of alkyl-substituted lactones represented in Tables 3 and 4 are of particular importance since many aldol reactions using alkyl ketones suffer from low stereoselectivities. Based on a previously reported carbonylation/ring-opening procedure, a variety of alkyl, ketone-aldol-type products were generated in near quantitative yields (Table 5). Epoxides were subjected to the corresponding carbonylation conditions shown in Tables 3 and 4 and purified before the addition of sodium methoxide in methanol. With kinetic resolutions that guarantee >99:1 e.r., this method is a desirable alternative to existing Mukaiyama aldol reactions, which often suffer from difficult ketone enantiofacial discrimination in addition to other drawbacks.

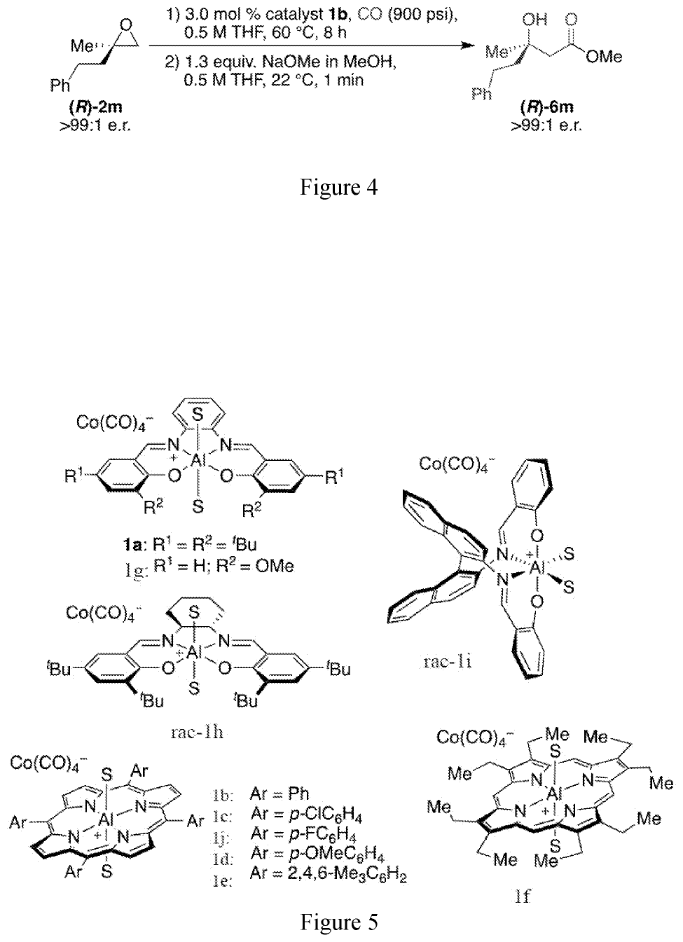

To test whether enantiopurity is maintained through our carbonylation and methanolysis procedures, an enantiopure substrate (R)-2m was synthesized in >99:1 e.r. using a kinetic resolution developed by Jacobsen and coworkers (FIG. 4). The corresponding aldol product was produced in a comparable yield to that of the racemic product (Table 5, entry 7) over two steps without loss of enantiopurity, indicating there was complete retention of stereochemistry.

Presented is an efficient and regioselective process for the carbonylation of 2,2-disubstituted epoxides using an octaethylporphyrin catalyst, 1f, and a tetraphenylporphyrin catalyst, 1b, although any of the catalysts presented are reasonable alternative catalysts. With optimized reaction conditions in hand, a large range of alkyl epoxides was carbonylated regioselectively. These epoxide substrates can be prepared in enantiopure form to produce enantiopure ketone-aldol-type products traditionally accessed via Mukaiyama aldol reactions. This sought-after tertiary alcohol functionality remains an important component of various natural products and pharmaceuticals. It is believed this is the first report of a carbonylative approach to the production of difficult-to-access ketone-aldol products.

General. Unless stated otherwise, all synthetic manipulations were carried out using standard Schlenk techniques under a nitrogen atmosphere or in an MBraun Unilab glovebox under an atmosphere of purified nitrogen. Reactions were carried out in oven-dried glassware cooled under vacuum. ¹H NMR and ³C{¹H} NMR spectra were recorded on a Bruker 500 MHz instrument at 22° C. with shifts reported relative to the residual solvent peak (CDCl₃: 7.26 ppm (¹H), and 77.16 ppm (³C)). Data are reported as follows: chemical shift, multiplicity (s=singlet, d=doublet, t=triplet, q=quartet, sep=septet, m=multiplet), integration, and coupling constants (Hz). Deuterated chloroform was purchased from Cambridge Isotope Laboratories and stored over 3 Å molecular sieves. HRMS analyses were performed on a Thermo Scientific Exactive Orbitrap MS system with an Ion Sense DART ion source. Enantiomeric ratios were determined by chiral HPLC of isolated material using a SHIMADZU system with a CHIRALPAK® OD column in comparison with authentic racemic materials. Optical rotations were measured on a Perkin-Elmer polarimeter in CHCl₃. The kinetics experiments were conducted on a Freeslate Core Module 3m (CM3) robotic platform located under a N2 atmosphere inside an MBraun drybox. The experiment was designed and executed using Library Studio® and Automation Studio® software. All solutions were dispensed robotically using a syringe dispense. These reactions were performed in a Freeslate HiP high pressure reactor containing 96×1 mL glass vials. For these experiments, solvent was removed in the glovebox using a vacuum centrifuge fitted with an external liquid nitrogen trap. Flash column chromatography was performed with silica gel (particle size 40-64 μm, 230-400 mesh) using mixtures of hexanes and diethyl ether. All work-up and purification procedures were carried out with reagent grade solvents (purchased from Fisher) in air.

TABLE 1
Activity and Regioselectivity of Catalysts 1a-1j for the Carbonylation
of Isobutylene Oxide

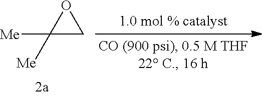

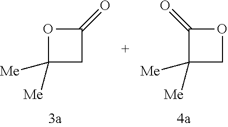 [SMILES: CC1(C)CC(=O)O1 ; CC1(C)COC1=O]

| | | | ratio |
|---|---|---|---|
| entry | catalyst | conv. (%)^{a} | 3a:4a^{a} |
| 1 | 1a | 24 | 94:6 |
| 2 | 1b | >99 | 93:7 |
| 3 | 1c | 97 | 93:7 |
| 4 | 1d | 82 | 93:7 |
| 5 | 1e | 62 | 93:7 |
| 6 | 1f | 78 | 96:4 |
| 7^{b} | 1g | 6 | 94:6 |
| 8^{b} | rac-1h | <1 | n.d. |
| 9^{b} | rac-1i | 6 | 61:39 |
| 10^{b} | 1j | 97 | 93:7 |

^{a}Determined by ¹H NMR analysis of crude reaction mixture.
^{b}2.0 mol % catalyst, 0.5 M 1,4-dioxane, 22° C., 18 h. n.d. = not determined.

TABLE 2
Effect of Solvent on Activity and Regioselectivity

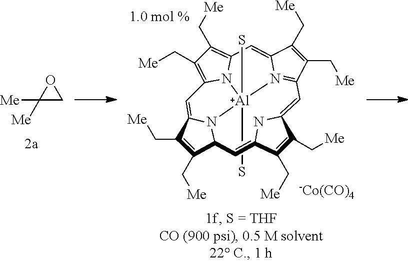

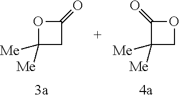 [SMILES: CC1(C)CC(=O)O1 ; CC1(C)COC1=O]

| entry | solvent | TOF (h⁻¹)^{a,b} | ratio 3a:4a^{a} |
|---|---|---|---|
| 1 | MeCN | 0 | n.d.^{c} |
| 2 | THF | 10 | 96:4 |
| 3 | 1,4-dioxane | 10 | 95:5 |
| 4 | DMTHF^{d} | 14 | >99:1 |
| 5 | Et₂O | 14 | 99:1 |
| 6 | ⁱPr₂O | 17 | >99:1 |
| 7 | toluene | 6 | 99:1 |
| 8 | hexanes | 3 | 94:6 |

^{a}Determined by ¹H NMR analysis of crude reaction mixture.
^{b}TOF = turnover frequency = (mol starting material consumed)/[(mol catalyst) • (trxn in hours)].
^{c}n.d. = not determined.
^{d}DMTHF = 2,5-dimethyltetrahydrofuran.

TABLE 3
Regioselective Carbonylation of Alkyl, 2,2-Disubstituted Epoxides

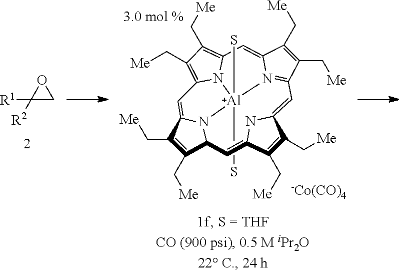

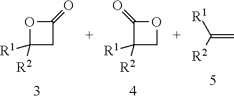

| | | ratio | isolated yield | alkene |
|---|---|---|---|---|
| entry | substrate | 3:4^{a} | 3 (%) | 5 (%)^{a} |
| 1 | 2a | 98:2 | 78^{b} | <1 |
| 2^{c} | 2b | >99:1 | 51^{b} | 2 |
| 3 | 2c | 98:2 | 66 | <1 |
| 4 | 2d | >99:1 | 79 | 2 |
| 5^{c} | 2e | >99:1 | 70 | <1 |
| 6^{e} | 2f | 98:2 | 79 | 2 |
| 7 | 2g | 98:2 | 88 | 2 |
| 8 | 2h | 98:2 | 46 | 3 |
| 9 | 2i | 98:2 | 78 | 2 |
| 10 | 2j | 99:1 | 67 | <1 |
| 11 | 2k | >99:1 | 91 | 3 |
| 12 | 2t | >99:1 | 47^{b} | 3 |

^{a}Determined by 1H NMR analysis of crude reaction mixture.
^{b}NMR yield versus hexamethyldisiloxane as an internal standard.
^{c}2.0 mol % catalyst 1b, 0.5 M dioxane, 40° C., 12 h.
^{d}Conversion determined by ¹H NMR analysis of crude reaction mixture.
^{e}5.0 mol % of 1f.

TABLE 4
Regioselective Carbonylation of Challenging 2,2-Disubstituted
Epoxides Using Catalyst 1b

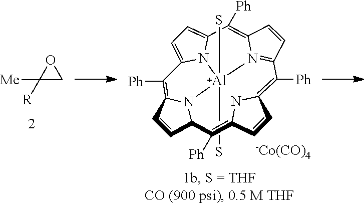

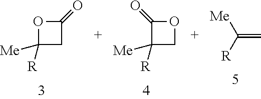

| | | | mol | | | | | |
|---|---|---|---|---|---|---|---|---|
| | | | % | | | | isolated | |
| en- | sub- | | cat- | temp. | time | ratio | yield | alkene^{a} |
| try | strate | R | alyst | (° C.) | (h) | 3:4^{a} | 3 (%) | 5 (%) |
| 1 | 2l | Bn | 2 | 60 | 6 | 98:2 | 67 | 2 |
| 2 | 2m | CH₂Bn | 3 | 60 | 8 | 99:1 | 51 | <1 |
| 3 | 2n | CH₂OPh | 3 | 60 | 8 | >99:1 | 81 | <1 |
| 4 | 2o | CH₂Cl | 3 | 40 | 4 | >99:1 | 84 | <1 |
| 5 | 2p | CH₂OTBS^{b} | 4 | 40 | 5 | 99:1 | 99 | <1 |
| 6 | 2q | CH₂CH₂OTBS^{b} | 3 | 40 | 5 | 96:4 | 91 | 4 |
| 7 | 2r | CO₂Me | 2 | 60 | 18 | 98:2 | 83 | <1 |
| 8 | 2s | CO₂^{t}Bu | 3 | 60 | 28 | 98:2 | 58 | <1 |

^{a}Determined by ¹H NMR analysis of crude reaction mixture.
^{b}TBS = tBuMe₂Si.

TABLE 5
Mukaiyama-Type Aldol Products Formed via the Methanolysis of β-
Lactones

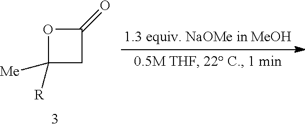

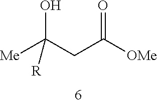

| entry | β-lactone | R | product | isolated yield 6 (%)^{a} |
|---|---|---|---|---|
| 1 | 3f | ⁿPent | 6f | 91 |
| 2 | 3l | Bn | 6l | 98 |
| 3 | 3m | CH₂Bn | 6m | 90 |
| 4 | 3n | CH₂OPh | 6n | 96 |
| 5 | 3q | CH₂CH₂OTBS | 6q | 98 |
| 6 | 3s | CO₂^{t}Bu | 6s | 85 |
| 7 | (R)-3m | CH₂Bn | (R)-6m | 100 |

^{a}See below for more details.

Sources of solvents, reagents, and catalysts:

Acetonitrile was passed through two columns packed with neutral alumina and copper(II) oxide and degassed by three freeze-pump-thaw cycles before being stored over 3 Å molecular sieves.

Azidotrimethylsilane was purchased from Sigma-Aldrich and used as received.

Calcium hydride was purchased from Strem and crushed with a mortar and pestle prior to use.

Carbon monoxide was purchased from Matheson (research quality/>99.99% min purity) and used as received.

2-(Chloromethyl)-2-methyloxirane (2o) was purchased from Combi-Blocks, dried over calcium hydride for 24 h, and degassed via three freeze-pump-thaw cycles prior to use.

3-Chloroperbenzoic acid was purchased from Sigma-Aldrich (577%) and used as received.

Cyclohexane was purchased from Fischer Chemical and used as received.

Deuterated chloroform was purchased from Cambridge Isotope Laboratories and stored over activated 3 Å molecular sieves.

Decolorizing carbon was purchased from Fischer Chemical and used as received.

Dichloromethane was purchased from Fischer Chemical and sparged vigorously with nitrogen for 40 minutes before passing through two packed columns of neutral alumina under nitrogen pressure. Later it was degassed via three freeze-pump-thaw cycles prior to use.

Dicobalt octacarbonyl was purchased from Strem and stored at −30° C. prior to use.

Diethyl ether was purchased from Fischer Chemical, dried over calcium hydride for 24 h, and degassed via three freeze-pump-thaw cycles prior to use.

Diethylaluminum chloride was purchased from Sigma-Aldrich and diluted to make a 1.0 M solution in dry hexanes prior to use.

Diisopropyl ether was purchased from TCI, dried over calcium hydride for 24 h, and degassed via three freeze-pump-thaw cycles prior to use.

2,3-Dimethyl-1-butene was purchased from TCI and used as received.

Dimethyl sulfoxide was purchased from Fisher Chemical and used as received.

2,5-Dimethyltetrahydrofuran was purchased from Lancaster Synthesis, dried over calcium hydride for 24 h, and degassed via three freeze-pump-thaw cycles prior to use.

1,4-Dioxane was purchased from Sigma-Aldrich, dried over 3 Å molecular sieves for 24 h, syringe filtered, and sparged with nitrogen for 30 minutes prior to use.

Hexamethyldisiloxane was purchased from Alfa Aesar, dried over 3 Å molecular sieves for 24 h, and degassed via three freeze-pump-thaw cycles prior to use.

Hexanes was purchased from Fischer Chemical and sparged with nitrogen for 40 minutes before passing through two packed columns of neutral alumina and copper(II) oxide under nitrogen pressure.

Imidazole was purchased from Sigma-Aldrich and used as received.

Isobutylene oxide (2a) was purchased from TCI, dried over calcium hydride for 24 h, and degassed via three freeze-pump-thaw cycles prior to use.

2,6-Lutidine was purchased from Sigma-Aldrich and used as received.

Magnesium sulfate was purchased from Fisher Chemical and used as received.

Methallylcyclopentane was purchased from Alfa Aesar and used as received.

Methallyl phenyl ether was purchased from Lancaster synthesis and used as received.

2-Methyl-1-butene was purchased from TCI and used as received.

3-Methyl-3-buten-1-ol was purchased from Lancaster Synthesis and used as received.

Methylenecyclohexane was purchased from Sigma-Aldrich and used as received.

2-Methyl-1-pentene was purchased from TCI and used as received.

2-Methyl-1-phenyl-1-propene was purchased from Alfa Aesar and used as received.

2-Methyl-2-propen-1-ol was purchased from Sigma-Aldrich and used as received.

2-Methyl-1-heptene was purchased from TCI and used as received.

Methyl methacrylate was purchased from Sigma-Aldrich and used as received.

2-Methyl-1-undecene was purchased from Sigma-Aldrich and used as received.

Octaethylporphyrin was purchased from Frontier Scientific and used as received.

4-Phenyl-2-butanone was purchased from TCI and used as received.

Potassium tert-butoxide was purchased from Sigma Aldrich and used as received.

2-Propanol was purchased from Fischer Chemical and dried over 3 Å molecular sieves for 24 h prior to use.

Sodium bicarbonate was purchased from Macron Fine Chemicals and diluted to make a saturated solution in deionized water prior to use.

Sodium hydroxide was purchased from J. T. Baker, crushed, and dried under vacuum prior to use.

Sodium methoxide solution in methanol (0.5 M) was purchased from Sigma-Aldrich and used as received.

Sodium thiosulfate was purchased from Alfa Aesar and diluted to make a saturated solution in deionized water prior to use.

Tert-butyldimethylchlorosilane was purchased from TCI and used as received.

Tert-butyldimethylsilyl trifluoromethanesulfonate was purchased from Alfa Aesar and used as received.

Tert-butyl methacrylate was purchased from TCI and used as received.

Tert-butyl 1-oxa-6-azaspiro[2.5]octane-6-carboxylate (2k) was purchased from Combi-Blocks and used as received.

Tetrahydrofuran was purchased from Fisher Chemical and sparged with nitrogen for 40 minutes before passing through two packed columns of neutral alumina and a third column packed with activated 4 Å molecular sieves under nitrogen pressure.

Toluene was purchased from Fisher Chemical and sparged with nitrogen for 40 minutes before passing through two packed columns of neutral alumina and copper(II) oxide under nitrogen pressure.

2,3,3-Trimethyl-1-butene was purchased from Sigma Aldrich and used as received.

Trimethylsulfoxonium iodide was purchased from TCI and used as received.

Control Experiment:

| 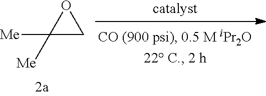 | | | |
|---|---|---|---|
| 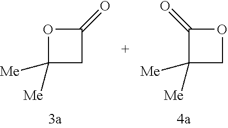 [SMILES: CC1(C)CC(=O)O1 ; CC1(C)COC1=O] | | | |
| entry | catalyst | mol % catalyst | conv. (%)^{a} |
| 1 | 1f | 1.0 | 45 |
| 2 | NaCo(CO)₄ | 1.0 | <1 |
| 3 | — | 0 | <1 |

^{a}Determined by 1H NMR analysis of crude reaction mixture.

Mechanistic Studies by ¹H NMR Analysis: Order in Carbon Monoxide. In a glovebox, 1f (1.0 mol %, 3.5 mg, 0.0040 mmol) was weighed into five individual 4 mL vials equipped with Teflon-coated magnetic stir bars and septa caps. A separate 8 mL glass vial equipped with a Teflon-coated magnetic stir bar was charged with 2a (186 mg, 2.60 mmol) and ⁱPr₂O (0.5 M, 5.2 mL). 0.8 mL of the stock solution was added to each of the five vials containing 1f. Each septum cap was pierced with a needle, and the vials were placed in a custom-made six-well high-pressure reactor. Each well was exposed to a different pressure of carbon monoxide (200, 350, 500, 700, and 900 psi). To work up these reactions, the reactor was carefully vented in a well-ventilated hood on emergency exhaust. A portion of each reaction was run through a decolorizing carbon plug with deuterated chloroform into an NMR tube to remove catalyst before analyzing by ¹H NMR spectroscopy. The conversion of 2a to 3a was plotted as a function of carbon monoxide pressure (P_{CO}). Assuming that [CO] is proportional to P_{CO}, this trend is consistent with a near zero-order rate dependence on P_{CO}.

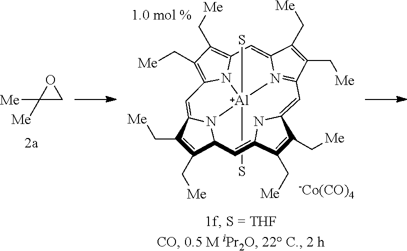

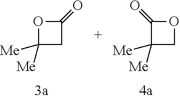 [SMILES: CC1(C)CC(=O)O1 ; CC1(C)COC1=O]

| entry | Pco (psi) | conv. (%)^{a} | ratio 3a:4a^{a} |
|---|---|---|---|
| 1 | 200 | 35 | >99:1 |
| 2 | 350 | 39 | >99:1 |
| 3 | 500 | 41 | 99:1 |
| 4 | 700 | 47 | 99:1 |
| 5 | 900 | 45 | 99:1 |

^{a}Determined by 1H NMR analysis of crude reaction mixture.

Mechanistic Studies by ¹H NMR Analysis: Order in Catalyst. These kinetics experiments were conducted on a Freeslate Core Module 3m (CM3) robotic platform under a N2 atmosphere in an MBraun drybox. These reactions were run in triplicate and performed in a Freeslate HiP high pressure reactor containing 96×1 mL glass vials. 100, 200, 300, 400, and 500 μl of a stock solution of 1f in Et₂O (10 mM) was dispensed robotically into individual 1 mL shell vials containing disposable stir bars. Volatiles were then removed from the vials using a Speedvac vacuum centrifuge (22° C., 30 minutes) fitted with an external liquid nitrogen trap. The vials were transferred to the Freeslate 96-well HiP reactor. The temperature of the reactor was set to −20° C., and the stir rate was set to 300 rpm. A stock solution of epoxide 2a (1.0 eq., 0.5 M) and an internal standard (hexamethyldisiloxane, 0.3 eq.) in diisopropyl ether (Pr₂O) was made and 200 μl was dispensed robotically into vials containing catalyst. The reactor was then sealed, brought out of the glovebox, and pressurized with 250 psi of carbon monoxide. The 15 vials were stopped after 2 h of reaction time by carefully venting the reactor in a well-ventilated hood on emergency exhaust. A portion of each reaction was run through a decolorizing carbon plug with deuterated chloroform into an NMR tube to remove catalyst before analyzing by ¹H NMR spectroscopy. The concentrations of lactone product were plotted as a function of initial catalyst concentration. The linear trend indicates that the reaction is first order in catalyst.

Mechanistic Studies by ¹H NMR Analysis: Order in Epoxide. These kinetics experiments were conducted on a Freeslate Core Module 3m (CM3) robotic platform under a N2 atmosphere in an MBraun drybox. These reactions were run in triplicate and performed in a Freeslate HiP high pressure reactor containing 96×1 mL glass vials. 300 μl of a stock solution of 1f in Et₂O (10 mM) was dispensed robotically into individual 1 mL shell vials containing disposable stir bars. Volatiles were then removed from the vials using a Speedvac vacuum centrifuge (22° C., 30 minutes) fitted with an external liquid nitrogen trap. The vials were transferred to the Freeslate 96-well HiP reactor. The temperature of the reactor was set to −20° C., and the stir rate was set to 300 rpm. 180, 160, 120, 80, 40, and 0 μl of ⁱPr₂O were dispensed into the six vials. A stock solution of 2a (1.0 M) and an internal standard (hexamethyldisiloxane, 0.3 eq.) in ⁱPr₂O was made. 20, 40, 80, 120, 160, and 200 μl of this stock solution were dispensed into each set of six vials containing 1f in ⁱPr₂O to generate 18 vials with 200 μl total volume (0.1, 0.2, 0.4, 0.6, 0.8, and 1.0 M). The reactor was then sealed, brought out of the glovebox, and pressurized with 250 psi of carbon monoxide. The 18 vials were stopped after 0.25 h of reaction time by carefully venting the reactor in a well-ventilated hood on emergency exhaust. A portion of each reaction was run through a decolorizing carbon plug with deuterated chloroform into an NMR tube to remove catalyst before analyzing by ¹H NMR spectroscopy. The concentrations of lactone product were plotted as a function of initial epoxide concentration. The shallow slope indicates that the reaction is zeroth order in epoxide using ⁱPr₂O as a solvent. These reactions were repeated in THF (for 2 h), generating a linear trend which indicates that the reaction is first order in epoxide using THF as a solvent.

General Procedure A: Epoxidation of Alkenes Using mCPBA (2a-2q).

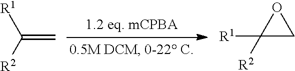

Under ambient atmosphere, 1.2 eq. of mCPBA (Aldrich, <77%) was added slowly to a stirring solution of a 1,1-disubstituted alkene in benchtop DCM (0.5 M) at 0° C. to avoid boiling. The resulting mixture was stirred at room temperature until complete consumption of starting material was observed by ¹H NMR of the crude reaction mixture. Aqueous Na₂S₂O₃ was added to destroy any excess mCPBA, and the reaction mixture stirred for an additional hour before filtering through celite. The aqueous phase was extracted with DCM (3×), and organics were combined and washed with NaHCO₃ (3×), dried with MgSO₄, filtered, and concentrated.

General Procedure B: Epoxidation of Methacrylates Using mCPBA (2r-2s).

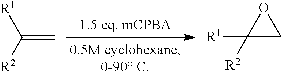

A literature procedure was adapted to for the epoxidation of methacrylate-type alkenes. Under ambient atmosphere, 1.5 eq. of mCPBA (Aldrich, <77%) was added slowly to a solution of a 1,1-disubstituted alkene in benchtop cyclohexane (0.5 M) at 0° C. to avoid boiling. The resulting mixture was stirred at 90° C. until complete consumption of starting material was observed by ¹H NMR of the crude reaction mixture. Aqueous Na₂S₂O₃ was added to destroy any excess mCPBA, and the reaction mixture stirred for an additional hour before filtering through celite. The filtrate was concentrated to 1/3 its original volume and filtered again to remove solids. The organic layer was washed with NaHCO₃ (3×) and H₂O (3×) before being dried with MgSO₄, filtered, and concentrated.

General Procedure C: Carbonylation of 2,2-disubstituted epoxides (3a-3s).

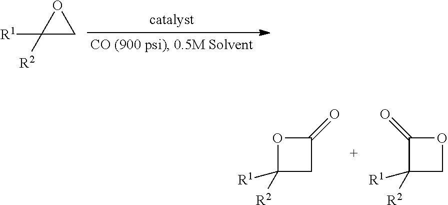

In a glovebox, a 4 mL glass vial equipped with a Teflon-coated magnetic stir bar and septum cap was charged with catalyst and solvent (0.5 M with respect to epoxide). The septum was then pierced with a needle, and the vial was placed in a custom-made six-well high-pressure reactor. The appropriate epoxide was then added dropwise to the vial by weight. The reactor was subsequently sealed, taken out of the glovebox, placed in a well-ventilated hood, and pressurized with carbon monoxide (900 psi). The closed reactor stirred for the time indicated. To work up these reactions, the reactor was carefully vented in a well-ventilated hood on emergency exhaust, and the crude reaction mixture was filtered through a decolorizing carbon plug made from a pipette and a glass microfiber filter to remove catalyst. The solution was concentrated and loaded with hexanes onto a column with increasing eluent polarity (100% hexanes, 19:1 hexanes:Et₂O, 9:1 hexanes:Et₂O, 3:1 hexanes:Et₂O). Both epoxide and lactone can be visualized using KMnO₄, cerium ammonium molybdate (CAM/Hanessian's Stain), and/or p-anisaldehyde TLC stains.

General Procedure D: Ring-opening of β,β-disubstituted β-lactones (6f, 6l, 6m, 6n, 6q, and 6s).

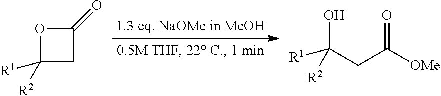

Purified lactone generated via general procedure C was diluted in THF (0.5 M) in a 20 mL vial before adding 1.3 eq. of NaOMe in MeOH. The mixture was stirred for 1 min before it was filtered through a silica gel plug, which was washed thoroughly with diethyl ether. The solution was concentrated to yield the aldol product. No column chromatography is necessary for purification.

General Procedure E: Alternative one-pot carbonylation/ring-opening method (6f, 6l, 6m, 6n, 6q, and 6s).

| 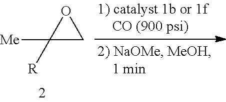 | | | | |
|---|---|---|---|---|
| 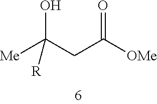 | | | | |
| | | | | yield 6 |
| entry | β-lactone | R | product | (%)^{a} |
| 1 | 2f | ⁿPent | 6f | 25 |
| 2 | 2l | Bn | 6l | 40 |
| 3 | 2m | CH₂Bn | 6m | 38 |
| 4 | 2n | CH₂OPh | 6n | 33 |
| 5 | 2q | CH₂CH₂OTBS | 6q | 72 |
| 6 | 2s | COO^{t}Bu | 6s | 54 |

^{a}Isolated yield after one-pot, two-step carbonylation/ring-opening reaction.

After the six-well high-pressure reactor was vented (see general procedure C) and before filtering with decolorizing carbon, the vial of crude lactone was charged with 1.3 eq. of 0.5 M NaOMe in MeOH and allowed to stir for 1 min. The crude mixture was then filtered through a decolorizing carbon pipette plug to remove the catalyst. After washing the plug with diethyl ether, the solution was concentrated and loaded with hexanes onto a column with increasing eluent polarity (100% hexanes, 19:1 hexanes:Et₂O, 9:1 hexanes:Et₂O). All aldol products can be visualized using KMnO₄, cerium ammonium molybdate (CAM/Hanessian's Stain), and/or p-anisaldehyde TLC stains.

### 2-Ethyl-2-methyloxirane (2b)

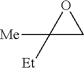

Following general procedure A, 2b was produced as a colorless oil (638 mg, 8% due to volatility). ¹H NMR (500 MHz, CDCl₃): δ 2.62 (d, 1H, J=4.9 Hz), 2.57 (d, 1H, J=4.9 Hz), 1.66-1.51 (m, 2H), 1.30 (s, 3H), 0.95 (t, 3H, J=7.6 Hz); ¹³C NMR (125 MHz, CDCl₃): δ 57.91, 53.75, 29.66, 20.72, 9.41; HRMS (DART): Calcd for C₅H₁₀O [M+H]⁺: 87.08044. Found: 87.08085.

### 2-Propyl-2-methyloxirane (2c)

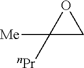

Following general procedure A, 2c was produced as a colorless oil (611 mg, 9% due to volatility). ¹H NMR (500 MHz, CDCl₃): δ 2.61 (d, 1H, J=4.9 Hz), 2.57 (d, 1H, J=4.9 Hz), 1.61-1.40 (m, 4H), 1.30 (s, 3H), 0.93 (t, 3H, J=7.3 Hz); ¹³C NMR (125 MHz, CDCl₃): δ 57.11, 54.09, 39.01, 20.99, 18.71, 14.29; HRMS (DART): Calcd for C₆H₁₂O [M+H]⁺: 101.09609. Found: 101.09638.

### 2-Isopropyl-2-methyloxirane (2d)

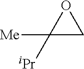

Following genera procedure A, 2d was produced as a colorless oil (6.77 g, 49%). ¹H NMR (500 MHz, CDCl₃): δ 2.60 (d, 1H, J=4.9 Hz), 2.55 (d, 1H, J=4.9 Hz), 1.50 (sep, 1H, J=6.9 Hz), 1.23 (s, 3H), 0.99 (d, 3H, J=6.9 Hz), 0.93 (d, 3H, J=7.0 Hz); ¹³C NMR (125 MHz, CDCl₃): δ 60.53, 53.87, 34.48, 18.65, 18.16, 17.15; HRMS (DART): Calcd for C₆H₁₂O [M+H]⁺: 101.09609. Found: 101.09647.

### 2-(Tert-butyl)-2-methyloxirane (2e)

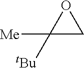

Following general procedure A, 2e was produced as a colorless oil (1.04 g, 18%). ¹H NMR (500 MHz, CDCl₃): δ 2.80 (d, 1H, J=4.7 Hz), 2.43 (d, 1H, J=4.7 Hz), 1.29 (s, 3H), 0.94 (s, 9H); ¹³C NMR (125 MHz, CDCl₃): δ 61.94, 51.89, 33.42, 26.12, 18.69; HRMS (DART): Calcd for C₇H₁₄O [M+H]⁺: 115.07536. Found: 115.07568.

### 2-Methyl-2-pentyloxirane (2f)

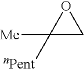

Following general procedure A, 2f was produced as a colorless oil (3.23 g, 79%). Analytical data match literature values. ¹H NMR (500 MHz, CDCl₃): δ 2.60 (d, 1H, J=4.9 Hz), 2.57 (d, 1H, J=4.9 Hz), 1.62-1.56 (m, 1H), 1.50-1.45 (m, 1H), 1.42-1.26 (m, 9H), 0.89 (t, 3H, J=6.8 Hz); ¹³C NMR (125 MHz, CDCl₃): δ 57.25, 54.12, 36.88, 32.01, 25.09, 22.75, 22.05, 14.15; HRMS (DART): Calcd for C₈H₁₆O [M+H]⁺: 129.12739. Found: 129.12744.

### 2-Hexyl-2-methyloxirane (2g)

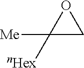

Following general procedure A, 2g was produced as a colorless oil (3.01 g, 57%). ¹H NMR (500 MHz, CDCl₃): δ 2.60 (d, 1H, J=5.0 Hz), 2.57 (d, 1H, J=4.9 Hz), 1.62-1.55 (m, 1H), 1.51-1.45 (m, 1H), 1.41-1.35 (m, 2H), 1.30-1.27 (m, 9H), 0.90-0.87 (m, 3H); ¹³C NMR (125 MHz, CDCl₃): δ 57.25, 54.12, 36.92, 31.94, 29.49, 25.38, 22.72, 21.05, 14.22; HRMS (DART): Calcd for C₉H₁₈O [M+H]⁺: 143.14304. Found: 143.14320.

### 2-Methyl-2-nonyloxirane (2h)

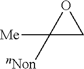

Following general procedure A, 2h was produced as a colorless oil (1.50 g, 73%). ¹H NMR (500 MHz, CDCl₃): δ 2.60 (d, 1H, J=5.0 Hz), 2.57 (d, 1H, J=4.9 Hz), 1.62-1.55 (m, 2H), 1.50-1.45 (m, 1H), 1.41-1.35 (m, 2H), 1.32-1.26 (m, 14H), 0.88 (t, 3H, J=6.9 Hz); ¹³C NMR (125 MHz, CDCl₃): δ 57.26, 54.13, 36.92, 32.04, 29.83, 29.74, 29.68, 29.46, 25.43, 22.83, 21.06, 14.27; HRMS (DART): Calcd for C₁₂H₂₄O [M+H]⁺: 185.18999. Found: 185.19074.

### 2-(Cyclopentylmethyl)-2-methyloxirane (2i)

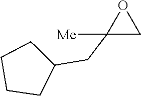

Following general procedure A, 2i was produced as a colorless oil (2.46 g, 68%). ¹H NMR (500 MHz, CDCl₃): δ 2.60 (d, 1H, J=5.0 Hz), 2.57 (d, 1H, J=4.9 Hz), 1.96-1.87 (m, 1H), 1.85-1.76 (m, 2H), 1.71 (dd, 1H, J=6.7, 13.8 Hz), 1.66-1.57 (m, 2H), 1.57-1.47 (m, 2H), 1.43 (dd, 1H, J=8.0, 13.8 Hz), 1.32 (s, 3H), 1.19-1.06 (m, 2H); ¹³C NMR (125 MHz, CDCl₃): δ 57.01, 54.33, 43.05, 37.24, 33.36, 33.02, 25.19, 25.03, 21.32; HRMS (DART): Calcd for C₉H₁₆O [M+H]⁺: 141.12739. Found: 141.12760.

### 1-Oxaspiro[2.5]octane (2j)

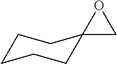

Following general procedure A, 2j was produced as a colorless oil (1.17 g, 20%). Analytical data match literature values ¹H NMR (500 MHz, CDCl₃): δ 2.59 (s, 2H), 1.76-1.71 (m, 2H), 1.60-1.48 (m, 8H); ¹³C NMR (125 MHz, CDCl₃): δ 59.19, 54.66, 33.81, 25.42, 25.06; HRMS (DART): Calcd for C₇H₁₂O [M+H]⁺: 113.09609. Found: 113.09638.

### 2-Benzyl-2-methyloxirane (2l)

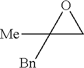 [SMILES: CC1(Cc2ccccc2)CO1]

Following general procedure A, 2l was produced as a colorless oil (4.92 g, 99%). ¹H NMR (500 MHz, CDCl₃): δ 7.35-7.31 (m, 2H), 7.28-7.24 (m, 3H), 2.92 (d, 1H, J=14.2 Hz), 2.85 (d, 1H, J=14.2 Hz), 2.68 (d, 1H, J=4.9 Hz), 2.64 (d, 1H, J=4.9 Hz), 1.31 (s, 3H); ¹³C NMR (125 MHz, CDCl₃): δ 137.31, 129.65, 128.42, 126.66, 57.35, 53.41, 43.19, 20.98; HRMS (DART): Calcd for C₁₀H₁₂O [M+H]⁺: 149.09609. Found: 149.09615.

### (R)-2-Methyl-2-phenethyloxirane (R-2m)

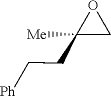 [SMILES: C[C@@]1(CCc2ccccc2)CO1]

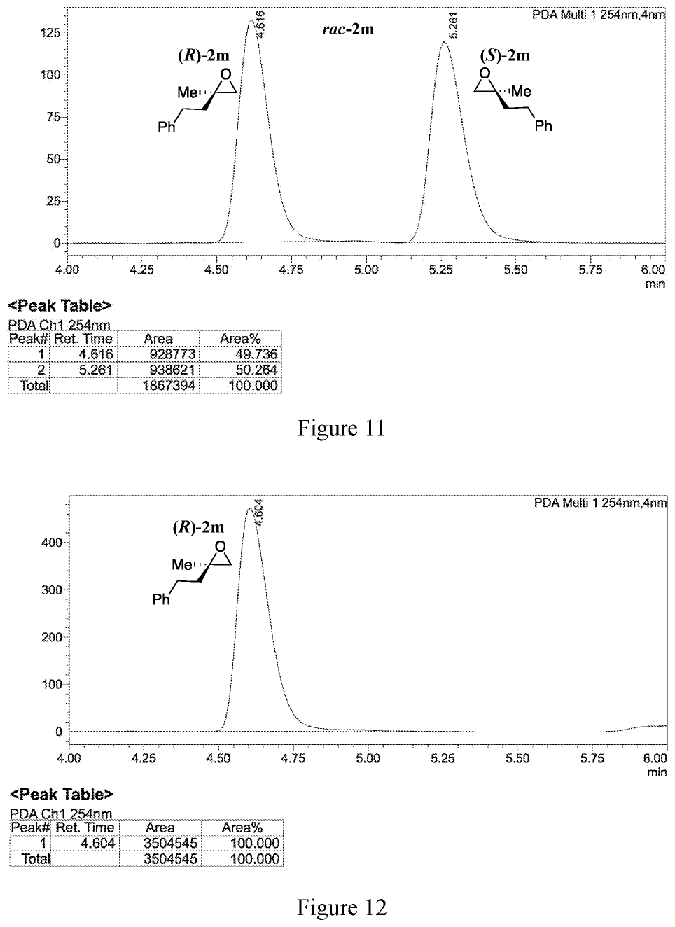

The title compound was synthesized using a procedure adapted from the literature. To a solution of KOⁱBu (1.00 eq., 7.10 g, 63.3 mmol) in DMSO (70 mL, 1.0 M) at 22° C. was added trimethylsulfoxonium iodide (1.10 eq., 15.3 g, 69.6 mmol), and the solution was allowed to stir for 30 min. A solution of 4-phenyl-2-butanone (1.00 eq., 63.3 mmol, 9.40 g) in DMSO (16 mL, 4.0 M) was added and allowed to stir until complete consumption of starting material was observed by ¹H NMR (approximately 30 min). The reaction mixture was diluted with ethyl acetate and water. The layers were separated, and the aqueous layer was extracted with ethyl acetate (3×). Combined organic layers were washed with brine (lx), dried with MgSO₄, filtered, and concentrated. The crude product was loaded with hexanes onto a column with increasing eluent polarity (100% hexanes, 19:1 hexanes:Et₂O, 9:1 hexanes:Et₂O), and 2m was produced as a colorless oil (7.34 g, 71%). A literature kinetic resolution procedure was used to obtain (R)-2m in >99:1 e.r. (1.54 g, 26%, maximum yield=40%) using 0.6 eq. of both ⁱPrOH and TMSN₃. ¹H NMR (500 MHz, CDCl₃): δ 7.30-7.27 (m, 2H), 7.21-7.18 (m, 3H), 2.77-2.67 (m, 2H), 2.61 (d, 1H, J=4.9 Hz), 2.58 (d, 1H, J=4.8 Hz), 1.96-1.90 (m, 1H), 1.87-1.81 (m, 1H), 1.38 (s, 3H); ¹³C NMR (125 MHz, CDCl₃): δ 141.74, 128.57, 128.41, 126.10, 56.85, 54.08, 38.71, 31.59, 21.19; HRMS (DART): Calcd for C₁₁H₁₄O [M+H]⁺: 163.11174. Found: 163.11166; specific rotation: [α]_{D}²⁰ −4.07 (c=0.54, CHCl₃) for an enantiomerically enriched sample of >99:1 e.r. Enantiomeric purity (>99:1) was determined by HPLC analysis (FIGS. 11 and 12) in comparison with authentic racemic material. CHIRALPAK® OD column, 90% hexanes, 10% ⁱPrOH, 1.0 mL/min, 254 nm.

### 2-Methyl-2-(phenoxymethyl)oxirane (2n)

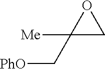 [SMILES: CC1(COc2ccccc2)CO1]

Following general procedure A, 2n was produced as a colorless oil (2.69 g, 26%). ¹H NMR (500 MHz, CDCl₃): δ 7.30-7.27 (m, 2H), 6.98-6.95 (m, 1H), 6.93-6.91 (m, 2H), 4.02 (d, 1H, J=10.4 Hz), 3.96 (d, 1H, J=10.9 Hz), 2.88 (d, 1H, J=4.8 Hz), 2.74 (d, 1H, J=4.8 Hz), 1.49 (s, 3H); ¹³C NMR (125 MHz, CDCl₃): δ 158.75, 129.63, 121.29, 114.78, 71.60, 55.70, 52.24, 18.67; HRMS (DART): Calcd for C₁₀H₁₂O₂ [M+H]⁺: 159.10157. Found: 159.10151.

### Tert-butyldimethyl((2-methyloxiran-2-yl)methoxy)silane (2p)

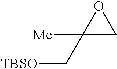 [SMILES: CC1(CO[Si](C)(C)C(C)(C)C)CO1]

Following general procedure A using S_{1, 2}p was produced as a colorless oil (7.47 g, 85%). ¹H NMR (500 MHz, CDCl₃): δ 3.65 (d, 1H, J=11.3 Hz), 3.60 (d, 1H, J=11.4 Hz), 2.74 (d, 1H, J=5.0 Hz), 2.60 (d, 1H, J=5.0 Hz), 1.34 (s, 3H), 0.90 (s, 9H), 0.07 (s, 3H), 0.06 (s, 3H); ¹³C NMR (125 MHz, CDCl₃): δ 66.71, 57.32, 51.81, 26.02, 18.51, 18.25, −5.23; HRMS (DART): Calcd for C₁₀H₂₂O₂Si [M+H]⁺: 203.14618. Found: 203.14613.

### Tert-butyldimethyl(2-(2-methyloxiran-2-yl)ethoxy)silane (R-2q)

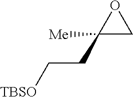 [SMILES: CC(C)(C)[Si](C)(C)OCC[C@]1(C)CO1]

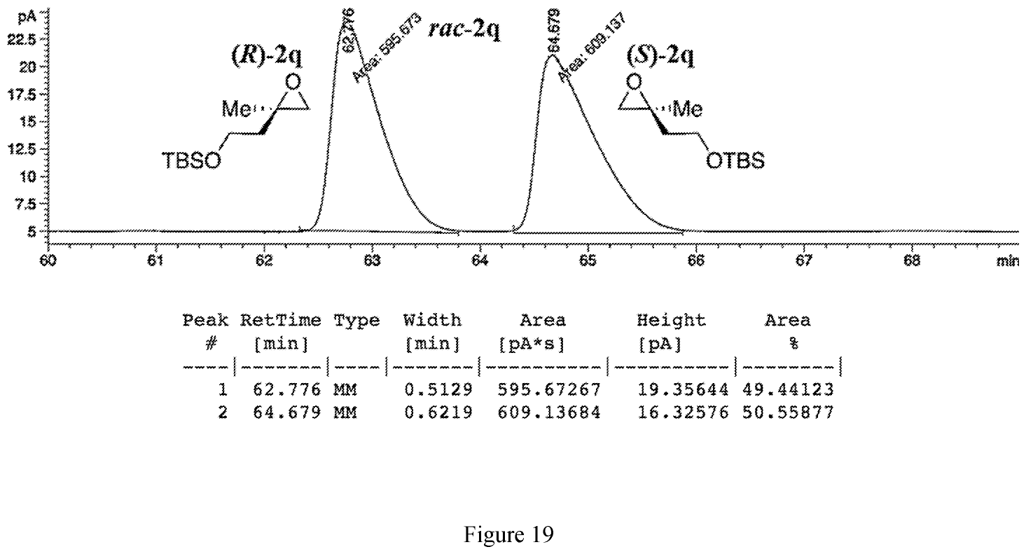

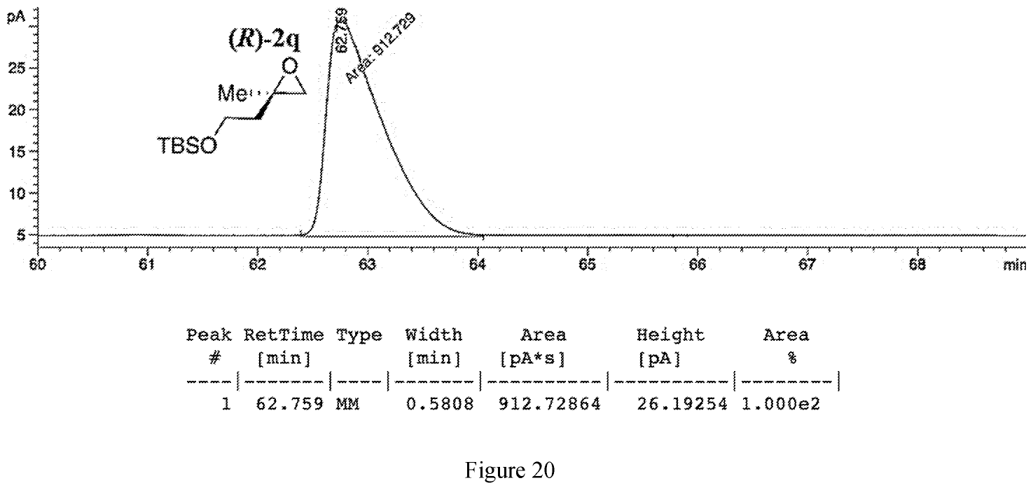

Following general procedure A using the corresponding alkene, 2q was produced as a colorless oil (17.7g, 89%). A kinetic resolution procedure (Lebel et al. Tet. Lett. 1999, 40, 7303-7306) was used to obtain (R)-2q in >99:1 e.r. (729 mg, 18%, maximum yield=40%) using 0.6 eq. of both ⁱPrOH and TMSN₃. ¹H NMR (500 MHz, CDCl₃): δ 3.76-3.69 (m, 2H), 2.69 (d, 1H, J=5.0 Hz), 2.58 (d, 1H, J=5.0 Hz), 1.89-1.84 (m, 1H), 1.72-1.66 (m, 1H), 1.34 (s, 3H), 0.89 (s, 9H), 0.05 (s, 3H), 0.05 (s, 3H); ¹³C NMR (125 MHz, CDCl₃): δ 59.84, 55.70, 54.29, 39.90, 26.02, 21.67, 18.34, −5.25, −5.27; HRMS (DART): Calcd for C₁₁H₂₄O₂Si [M+H]⁺: 217.16183. Found: 217.16185. specific rotation: [α]_{D}²⁰ −12.0 (c=0.37, CHCl₃) for an enantiomerically enriched sample of >99:1 e.r. Enantiomeric purity (>99:1) was determined by chiral GC analysis (using a Supelco β-Dex 225 column) in comparison with authentic racemic material (FIGS. 19 and 20).

### Methyl 2-methyloxirane-2-carboxylate (2r)

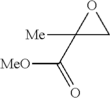

Following general procedure B, 2r was produced as a colorless oil (4.38 g, 40%). ¹H NMR (500 MHz, CDCl₃): δ 3.76 (s, 3H), 3.11 (d, 1H, J=6.1 Hz), 2.77 (d, 1H, J=6.0 Hz), 1.58 (s, 3H); ¹³C NMR (125 MHz, CDCl₃): δ 171.31, 53.86, 53.15, 52.77, 17.59; HRMS (DART): Calcd for C₅H₈O₃ [M+H]⁺: 117.05462. Found: 117.05497.

### Tert-butyl 2-methyloxirane-2-carboxylate (2s)

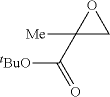 [SMILES: CC(C)(C)OC(=O)C1(C)CO1]

Following general procedure B, 2s was produced as a colorless oil (9.77 g, 50%). ¹H NMR (500 MHz, CDCl₃): δ 3.04 (d, 1H, J=6.1 Hz), 2.69 (d, 1H, J=6.2 Hz), 1.53 (s, 3H), 1.47 (s, 9H); ¹³C NMR (125 MHz, CDCl₃): δ 169.85, 82.24, 54.36, 53.01, 28.03, 17.67; HRMS (DART): Calcd for C₈H₁₄O₃ [M+H]⁺: 159.10157. Found: 159.10165.

### 2,2-Diethyloxirane (2t)

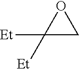

Following general procedure A, 2t was produced as a colorless oil (4.37 g, 36%). ¹H NMR (500 MHz, CDCl₃): δ 2.58 (s, 2H), 1.69-1.53 (m, 4H), 0.92 (t, 6H, J=7.6 Hz); ¹³C NMR (125 MHz, CDCl₃): δ 61.01, 51.97, 26.84, 9.04; HRMS (DART): Calcd for C₆H₁₃O [M+H]⁺: 101.09609. Found: 101.09649.

### 4,4-Dimethyloxetan-2-one (3a)

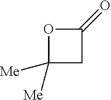

3a was produced in 78% yield (determined by ¹H NMR versus hexamethyldisiloxane as an internal standard) using general procedure C. Analytical data match literature values. ¹H NMR (500 MHz, CDCl₃): δ 3.16 (s, 2H), 1.59 (s, 6H); ¹³C NMR (125 MHz, CDCl₃): δ 167.48, 76.18, 48.64, 26.37.

### 4-Ethyl-4-methyloxetan-2-one (3b)

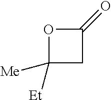

Following general procedure C, 3b was produced in 51% yield (determined by ¹H NMR versus hexamethyldisiloxane as an internal standard) as a colorless oil. ¹H NMR (500 MHz, CDCl₃): δ 3.18 (d, 1H, J=16.1 Hz), 3.10 (d, 1H, J=16.1 Hz), 1.93-1.81 (m, 2H), 1.57 (s, 3H), 1.02 (t, 3H, J=7.5 Hz); ¹³C NMR (125 MHz, CDCl₃): δ 168.30, 79.22, 47.02, 32.43, 23.96, 8.55; HRMS (DART): Calcd for C₆H₁₀O₂ [M+H]⁺: 115.07536. Found: 115.07565.

### 4-Methyl-4-propyloxetan-2-one (3c)

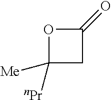

Following general procedure C, 3c was produced as a colorless oil (33.4 mg, 66%). ¹H NMR (500 MHz, CDCl₃): δ 3.18 (d, 1H, J=16.1 Hz), 3.10 (d, 1H, J=16.1 Hz), 1.87-1.75 (m, 2H), 1.57 (s, 3H), 1.50-0.98 (m, 2H), 0.98 (t, 3H, J=7.4 Hz); ¹³C NMR (125 MHz, CDCl₃): δ 168.36, 78.82, 47.62, 41.64, 24.38, 17.77, 14.23; HRMS (DART): Calcd for C₇H₁₂O₂ [M+H]⁺: 129.09101. Found: 129.09123.

### 4-Isopropyl-4-methyloxetan-2-one (3d)

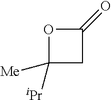

Following general procedure C, 3d was produced as a colorless oil (20.6 mg, 79%). ¹H NMR (500 MHz, CDCl₃): δ 3.17 (d, 1H, J=16.1 Hz), 3.02 (d, 1H, J=16.1 Hz), 2.05 (sep, 1H, J=6.9 Hz), 1.50 (s, 3H), 1.02 (d, 3H, J=6.8 Hz), 0.97 (d, 3H, J=7.0 Hz); ¹³C NMR (125 MHz, CDCl₃): δ 168.53, 81.58, 46.55, 36.23, 20.25, 17.30, 16.72; HRMS (DART): Calcd for C₇H₁₂O₂ [M+H]⁺: 129.09101. Found: 129.09109.

### 4-(Tert-butyl)-4-methyloxetan-2-one (3e)

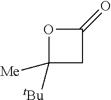

Following general procedure C, 3e was produced as a colorless oil (55.7 mg, 70%). ¹H NMR (500 MHz, CDCl₃): δ 3.37 (d, 1H, J=16.3 Hz), 2.92 (d, 1H, J=16.3 Hz), 1.58 (s, 3H), 1.04 (s, 9H); ¹³C NMR (125 MHz, CDCl₃): δ 168.70, 83.19, 44.59, 36.03, 24.63, 21.78; HRMS (DART): Calcd for C₈H₁₄O₂ [M+H]⁺: 143.10666. Found: 143.10686.

### 4-Methyl-4-pentyloxetan-2-one (3f)

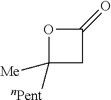

Following general procedure C, 3f was produced as a colorless oil (51.2 mg, 79%). ¹H NMR (500 MHz, CDCl₃): δ 3.18 (d, 1H, J=16.1 Hz), 3.10 (d, 1H, J=16.1 Hz), 1.88-1.76 (m, 2H), 1.57 (s, 3H), 1.44-1.29 (m, 6H), 0.92-0.88 (m, 3H); ¹³C NMR (125 MHz, CDCl₃): δ 168.38, 78.90, 47.58, 39.51, 31.86, 24.42, 24.06, 22.61, 14.07; HRMS (DART): Calcd for C₉H₁₆O₂ [M+H]⁺: 157.12231. Found: 157.12246.

### 4-Hexyl-4-methyloxetan-2-one (3g)

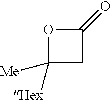

Following general procedure C, 3g was produced as a colorless oil (59.1 mg, 88%). ¹H NMR (500 MHz, CDCl₃): δ 3.18 (d, 1H, J=16.1 Hz), 3.10 (d, 1H, J=16.1 Hz), 1.88-1.76 (m, 2H), 1.57 (s, 3H), 1.41-1.29 (m, 8H), 0.91-0.88 (m, 3H); ¹³C NMR (125 MHz, CDCl₃): δ 168.39, 78.91, 47.58, 39.55, 31.76, 29.37, 24.43, 24.36, 22.66, 14.18; HRMS (DART): Calcd for C₁₀H₁₈O₂ [M+H]⁺: 171.13796. Found: 171.13813.

### 4-Methyl-4-nonyloxetan-2-one (3h)

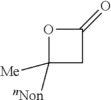

Following general procedure C, 3h was produced as a colorless oil (39.2 mg, 46%). ¹H NMR (500 MHz, CDCl₃): δ 3.18 (d, 1H, J=16.1 Hz), 3.10 (d, 1H, J=16.1 Hz), 1.88-1.76 (m, 2H), 1.57 (s, 3H), 1.44-1.27 (m, 14H), 0.88 (t, 3H, J=6.8 Hz); ¹³C NMR (125 MHz, CDCl₃): δ 168.39, 78.92, 47.58, 39.56, 32.00, 29.72, 29.60, 29.59, 29.41, 24.43, 24.40, 22.82, 14.26; HRMS (DART): Calcd for C₁₃H₂₄O₂ [M+H]⁺: 212.18491. Found: 213.18496.

### 4-(Cyclopentylmethyl)-4-methyloxetan-2-one (3i)

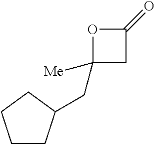 [SMILES: CC1(CC2CCCC2)CC(=O)O1]

Following general procedure C, 3i was produced as a colorless oil (66.1 mg, 78%). ¹H NMR (500 MHz, CDCl₃): δ 3.21 (d, 1H, J=16.1 Hz), 3.11 (d, 1H, J=16.1 Hz), 1.98-1.76 (m, 4H), 1.67-1.63 (m, 2H), 1.60 (s, 3H), 1.56-1.52 (m, 2H), 1.23-1.09 (m, 2H); ¹³C NMR (125 MHz, CDCl₃): δ 168.45, 79.08, 48.27, 45.14, 36.64, 33.59, 33.48, 25.12, 24.89, 24.86; HRMS (DART): Calcd for C₁₀H₁₆O₂ [M+H]⁺: 169.12231. Found: 169.12241.

### 1-Oxaspiro[3.5]nonan-2-one (3j)

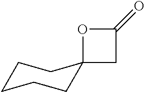

Following general procedure C, 3j was produced as a colorless oil (39.1 mg, 67%). ¹H NMR (500 MHz, CDCl₃): δ 3.07 (s, 2H), 1.92-1.88 (m, 2H), 1.82-1.72 (m, 4H), 1.49-1.44 (m, 4H); ¹³C NMR (125 MHz, CDCl₃): δ 168.69, 79.00, 47.46, 36.00, 24.71, 23.43; HRMS (DART): Calcd for C₈H₁₂O₂ [M+H]⁺: 141.09101. Found: 141.09109.

### Tert-butyl 2-oxo-1-oxa-7-azaspiro[3.5]nonane-7-carboxylate (3k)

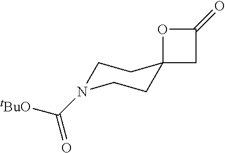 [SMILES: CC(C)(C)OC(=O)N1CCC2(CC1)CC(=O)O2]

Following general procedure C, 3k was produced as a white solid (91.0 mg, 91%). ¹H NMR (500 MHz, CDCl₃): δ 3.74-3.72 (m, 2H), 3.34 (ddd, 2H, J=3.6, 9.5, 13.4 Hz), 3.17 (s, 2H), 1.96-1.85 (m, 4H), 1.47 (s, 9H); ¹³C NMR (125 MHz, CDCl₃): δ 167.42, 154.64, 80.28, 76.55, 47.47, 40.65, 35.20, 28.53; HRMS (DART): Calcd for C₁₂H₁₉NO₄ [M+H]⁺: 242.13868. Found: 242.13886.

### 4-Benzyl-4-methyloxetan-2-one (3l)

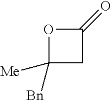 [SMILES: CC1(Cc2ccccc2)CC(=O)O1]

Following general procedure C, 3l was produced as a colorless oil (22.5 mg, 67%). ¹H NMR (500 MHz, CDCl₃): δ 7.35-7.27 (m, 3H), 7.23 (app. d, 2H, J=7.3 Hz), 3.25 (d, 1H, J=16.2 Hz), 3.14 (d, 1H, J=14.3 Hz), 3.10 (d, 1H, J=14.3 Hz), 3.09 (d, 1H, J=16.3 Hz), 1.58 (s, 3H); ¹³C NMR (125 MHz, CDCl₃): δ 167.60, 135.23, 130.15, 128.76, 127.49, 78.11, 47.03, 45.28, 24.92; HRMS (DART): Calcd for C₁₁H₁₂O₂ [M+H]⁺: 177.09101. Found: 177.09121.

### (R)₄-Methyl-4-phenethyloxetan-2-one (R-3m)

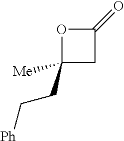 [SMILES: C[C@@]1(CCc2ccccc2)CC(=O)O1]

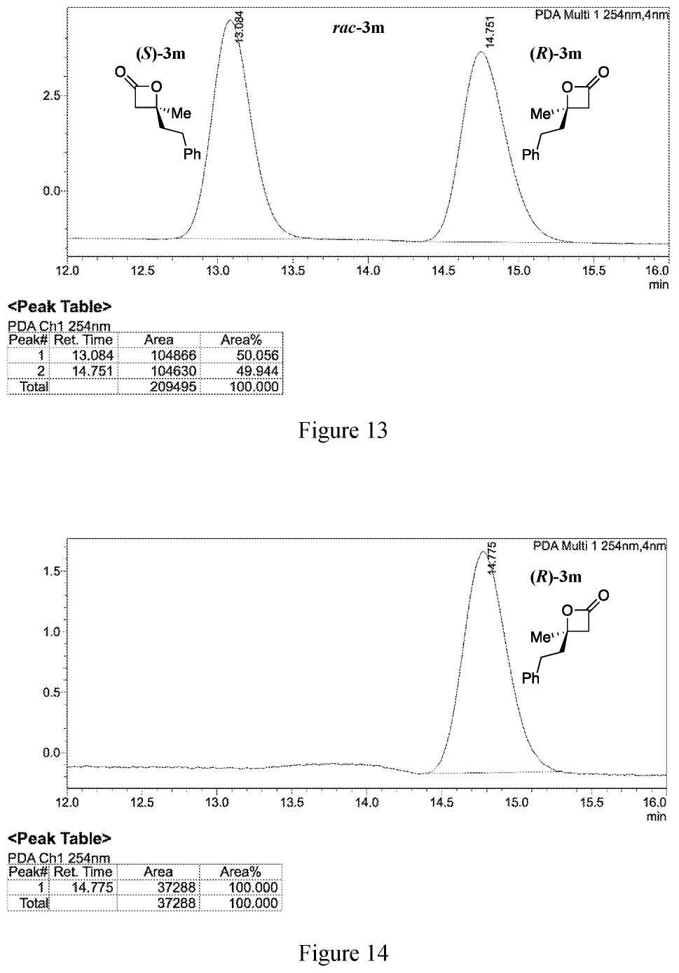

Following general procedure C using (R)-2m, (R)-3m was produced as a colorless oil (for enantioenriched: 27.7 mg, 42%; for rac: 38.3 mg, 51%). ¹H NMR (500 MHz, CDCl₃): δ 7.31 (app. t, 2H, J=7.3 Hz), 7.24-7.19 (m, 3H), 3.17 (d, 1H, J=16.2 Hz), 3.11 (d, 1H, J=16.2 Hz), 2.82-2.69 (m, 2H), 2.22-2.11 (m, 2H), 1.65 (s, 3H); ¹³C NMR (125 MHz, CDCl₃): δ 168.03, 140.58, 128.82, 128.37, 126.52, 78.35, 47.74, 41.32, 30.66, 24.49; HRMS (DART): Calcd for C₁₂H₁₄O₂ [M+H]⁺: 191.10666. Found: 191.10669. specific rotation: [α]_{D}²⁰+3.21 (c=0.53, CHCl₃) for an enantiomerically enriched sample of >99:1 e.r. Enantiomeric purity (>99:1) was determined by HPLC analysis (FIGS. 13 and 14) in comparison with authentic racemic material. CHIRALPAK® OD column, 90% hexanes, 10%ⁱPrOH, 1.0 mL/min, 254 nm.

### 4-Methyl-4-(phenoxymethyl)oxetan-2-one (3n)

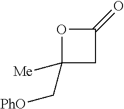 [SMILES: CC1(COc2ccccc2)CC(=O)O1]

Following general procedure C, 3n was produced as a colorless oil (61.7 mg, 81%). ¹H NMR (500 MHz, CDCl₃): δ 7.32-7.29 (m, 2H), 7.02-6.99 (m, 1H), 6.94-6.92 (m, 2H), 4.19 (d, 1H, J=10.5 Hz), 4.11 (d, 1H, J=10.5 Hz), 3.64 (d, 1H, J=16.0 Hz), 3.21 (d, 1H, J=16.1 Hz), 1.71 (s, 3H); ¹³C NMR (125 MHz, CDCl₃): δ 167.08, 158.22, 129.77, 121.88, 114.84, 77.41, 71.00, 45.25, 22.15; HRMS (DART): Calcd for C₁₁H₁₂O₃ [M+H]⁺: 193.08592. Found: 193.08605.

### 4-(Chloromethyl)-4-methyloxetan-2-one (30)

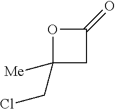

Following general procedure C, 3o was produced as a colorless oil (22.7 mg, 84%). ¹H NMR (500 MHz, CDCl₃): δ 3.78-3.73 (m, 2H), 3.49 (d, 1H, J=16.4 Hz), 3.22 (d, 1H, J=16.4 Hz), 1.71 (s, 3H); ¹³C NMR (125 MHz, CDCl₃): δ 166.28, 77.41, 48.84, 46.59, 22.73; HRMS (DART): Calcd for C₅H₇ClO₂ [M+H]⁺: 135.02073. Found: 135.02097.

### 4-(((Tert-butyldimethylsilyl)oxy)methyl)-4-methyloxetan-2-one (3p)

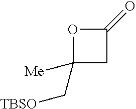 [SMILES: CC1(CO[Si](C)(C)C(C)(C)C)CC(=O)O1]

Following general procedure C, 3p was produced as a colorless oil (45.5 mg, 99%). ¹H NMR (500 MHz, CDCl₃): δ 3.81 (d, 1H, J=11.7 Hz), 3.69 (d, 1H, J=11.7 Hz), 3.48 (d, 1H, J=15.8 Hz), 3.00 (d, 1H, J=15.8 Hz), 1.54 (s, 3H), 0.90 (s, 9H), 0.08 (s, 6H); ¹³C NMR (125 MHz, CDCl₃): δ 167.71, 77.85, 66.38, 43.75, 25.83, 25.80, 21.33, 18.32; HRMS (DART): Calcd for C₁₁H₂₂O₃Si [M+H]⁺: 231.14110. Found: 231.14125.

### (R)-4-(2-((Tert-butyldimethylsilyl)oxy)ethyl)-4-methyloxetan-2-one (R-3q)

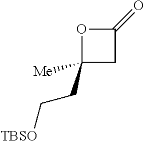 [SMILES: CC(C)(C)[Si](C)(C)OCC[C@]1(C)CC(=O)O1]

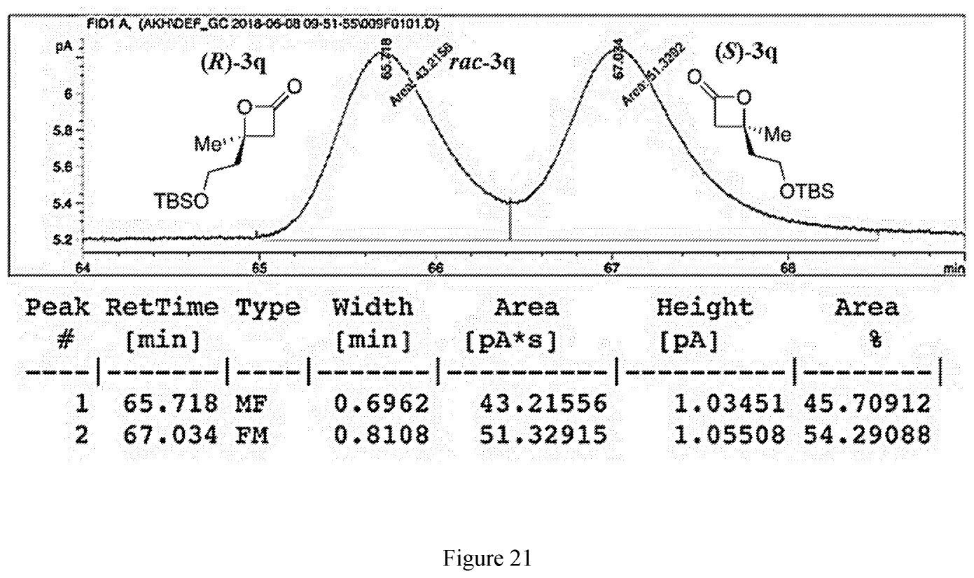

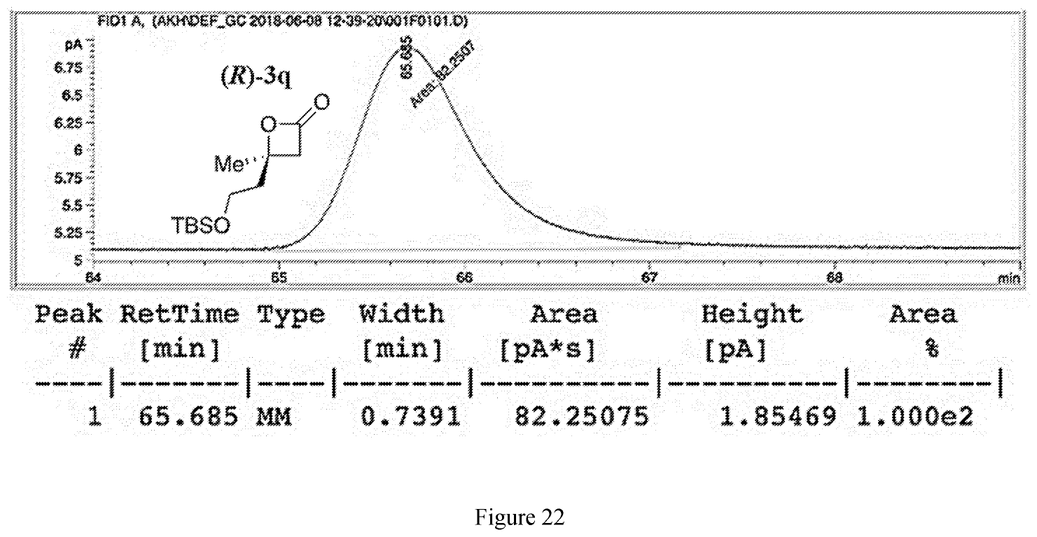

Following general procedure C using (R)-2q, (R)-3q was produced as a colorless oil (for rac: 89.3 mg, 91%). ¹H NMR (500 MHz, CDCl₃): δ 3.84-3.81 (m, 1H), 3.78-3.75 (m, 1H), 3.51 (d, 1H, J=16.3 Hz), 3.10 (d, 1H, J=16.3 Hz), 2.09-2.05 (m, 2H), 1.61 (s, 3H), 0.88 (s, 9H), 0.06 (s, 3H), 0.05 (s, 3H); ¹³C NMR (125 MHz, CDCl₃): δ 168.61, 77.97, 59.08, 48.39, 41.30, 25.97, 24.98, 18.27, −5.39, −5.44; HRMS (DART): Calcd for C₁₂H₂₄O₃Si [M+H]⁺: 245.15675. Found: 245.15676. specific rotation: [α]_{D}²⁰ −9.93 (c=0.26, CHCl₃) for an enantiomerically enriched sample of >99:1 e.r. Enantiomeric purity (>99:1) was determined by chiral GC analysis (using an Astec CHIRALDEX A-TA column) in comparison with authentic racemic material (FIGS. 21 and 22).

### Methyl 2-methyl-4-oxooxetane-2-carboxylate (3r)

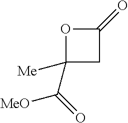

Following general procedure C, 3r was produced as a colorless oil (23.9 mg, 83%). ¹H NMR (500 MHz, CDCl₃): δ 3.84 (s, 3H), 3.75 (d, 1H, J=16.4 Hz), 3.36 (d, 1H, J=16.4 Hz), 1.81 (s, 3H); ¹³C NMR (125 MHz, CDCl₃): δ 170.26, 165.85, 74.04, 53.38, 48.39, 22.08; HRMS (DART): Calcd for C₆H₈O₄ [M+H]⁺: 145.04954. Found: 145.04976.

### Tert-butyl 2-methyl-4-oxooxetane-2-carboxylate (3s)

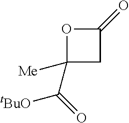 [SMILES: CC(C)(C)OC(=O)C1(C)CC(=O)O1]

Following general procedure C, 3s was produced as a colorless oil (21.6 mg, 58%). ¹H NMR (500 MHz, CDCl₃): δ 3.69 (d, 1H, J=16.4 Hz), 3.30 (d, 1H, J=16.3 Hz), 1.77 (s, 3H), 1.51 (s, 9H); ¹³C NMR (125 MHz, CDCl₃): δ 168.82, 166.43, 83.77, 74.54, 48.04, 27.94, 22.00; HRMS (DART): Calcd for C₉H₁₆O₂ [M+H]⁺: 187.09649. Found: 187.09666.

### 4,4-Diethyloxetan-2-one (3t)

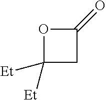

Following general procedure C, 3t was produced in 47% yield (determined by ¹H NMR versus hexamethyldisiloxane as an internal standard) as a colorless oil. ¹H NMR (500 MHz, CDCl₃): δ 3.08 (s, 2H), 1.93-1.80 (m, 4H), 0.98 (t, 6H, J=7.5 Hz); ¹³C NMR (125 MHz, CDCl₃): δ 168.60, 81.64, 44.87, 29.35, 8.05; HRMS (DART): Calcd for C₇H₁₃O₂ [M+H]⁺: 129.09101. Found: 129.09113.

### Methyl 3-hydroxy-3-methyloctanoate (6f)

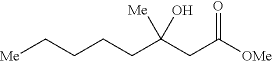 [SMILES: CCCCCC(C)(O)CC(=O)OC]

Following general procedure D, 6f was produced as a colorless oil (56.6 mg, 91%). ¹H NMR (500 MHz, CDCl₃): δ 3.71 (s, 3H), 3.41 (s, 1H), 2.52 (d, 1H, J=15.6 Hz), 2.44 (d, 1H, J=15.6 Hz), 1.51-1.48 (m, 2H), 1.37-1.25 (m, 6H), 1.23 (s, 3H), 0.89 (app. t, 3H); ¹³C NMR (125 MHz, CDCl₃): δ 173.68, 71.15, 51.80, 44.80, 42.19, 32.40, 26.82, 23.77, 22.74, 14.17; HRMS (DART): Calcd for C₁₀H₂₀O₃ [M+H]⁺: 189.14852. Found: 189.14847.

### Methyl 3-hydroxy-3-methyl-4-phenylbutanoate (6l)

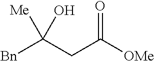 [SMILES: COC(=O)CC(C)(O)Cc1ccccc1]

Following general procedure D, 6l was produced as a colorless oil (29.3 mg, 98%). ¹H NMR (500 MHz, CDCl₃): δ 7.31-7.28 (m, 2H), 7.24-7.21 (m, 3H), 3.70 (s, 3H), 2.89-2.82 (m, 2H), 2.49 (d, 1H, J=15.8 Hz), 2.43 (d, 1H, J=15.8 Hz), 1.25 (s, 3H); ¹³C NMR (125 MHz, CDCl₃): δ 173.48, 137.25, 130.67, 128.31, 126.75, 71.36, 51.85, 48.19, 44.19, 27.28; HRMS (DART): Calcd for C₁₂H₁₆O₃ [M+H]⁺: 209.11722. Found: 209.11717.

### (R)-Methyl 3-hydroxy-3-methyl-5-phenylpentanoate (6m)

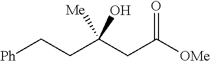 [SMILES: COC(=O)C[C@](C)(O)CCc1ccccc1]

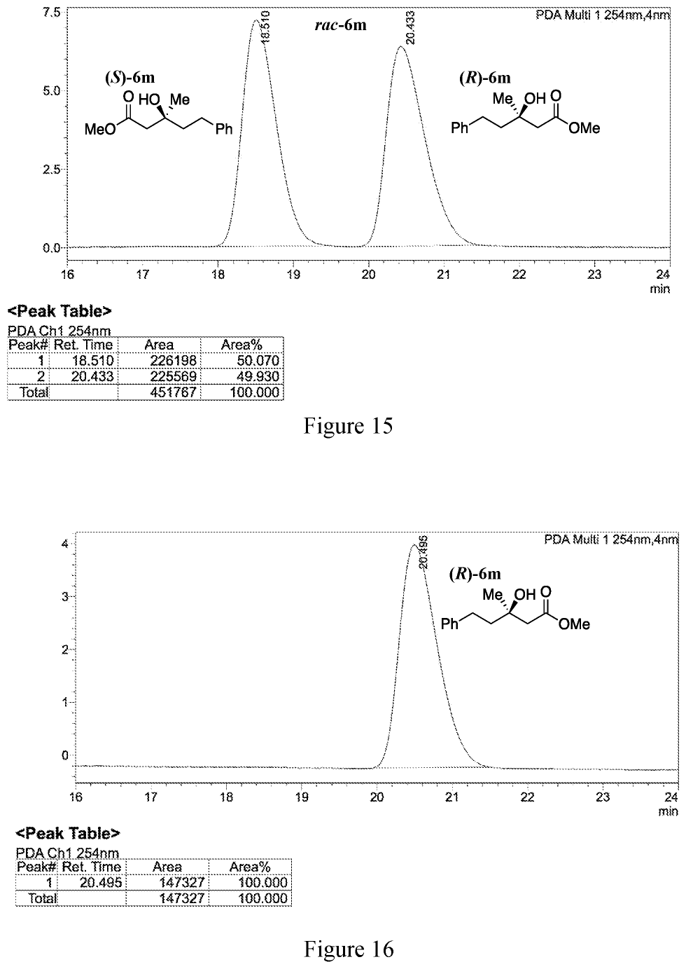

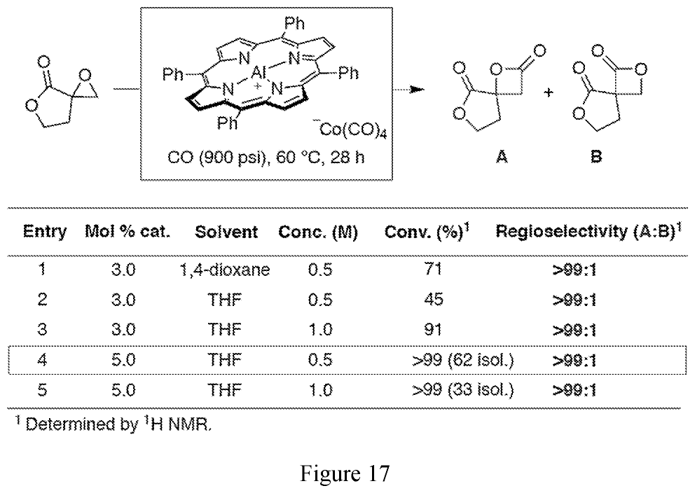

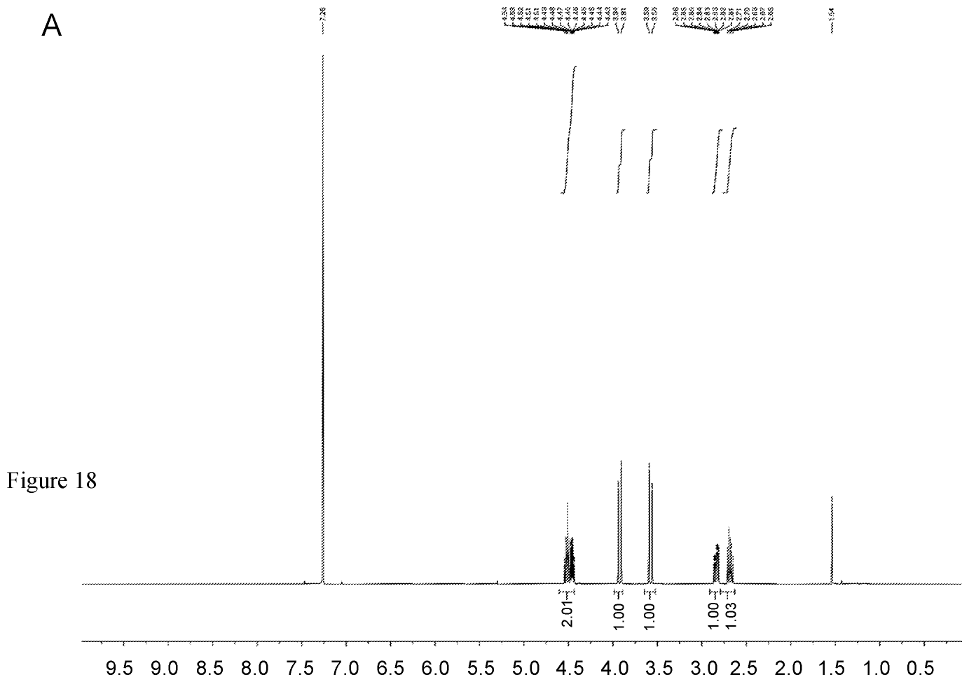

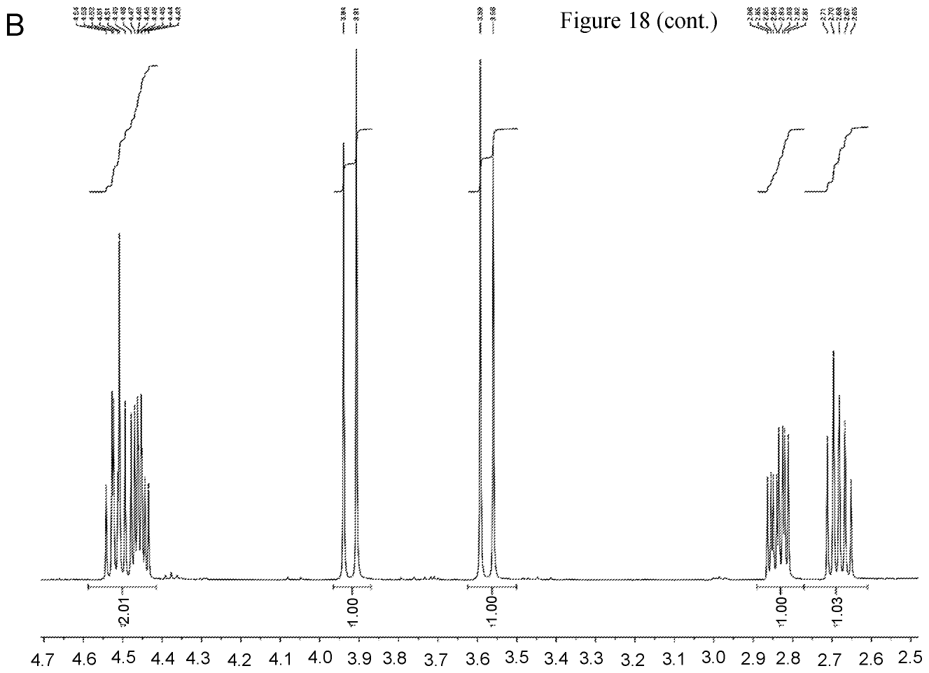

Following general procedure D using (R)-3m, (R)-6m was produced as a colorless oil (for enantioenriched: 32.5 mg, 100%; for rac: 40.0 mg, 90%). ¹H NMR (500 MHz, CDCl₃): δ 7.28 (app. t, 2H, J=7.8 Hz), 7.20-7.17 (m, 3H), 3.72 (s, 3H), 3.54 (s, 1H), 2.77-2.66 (m, 2H), 2.59 (d, 1H, J=15.7 Hz), 2.51 (d, 1H, J=15.7 Hz), 1.88-1.78 (m, 2H), 1.32 (s, 3H); ¹³C NMR (125 MHz, CDCl₃): δ 173.57, 142.33, 128.57, 128.48, 125.97, 70.93, 51.89, 44.94, 43.98, 30.42, 26.84; HRMS (DART): Calcd for C₁₃H₁₈O₃ [M+H]⁺: 223.13287. Found: 223.13280. specific rotation: [α]_{D}²⁰ −4.95 (c=0.47, CHCl₃) for an enantiomerically enriched sample of >99:1 e.r. Enantiomeric purity (>99:1) was determined by HPLC analysis in comparison with authentic racemic material (FIGS. 15 and 16). CHIRALPAK® OD column, 98% hexanes, 2% ⁱPrOH, 1.0 mL/min, 254 nm.

### Methyl 3-hydroxy-3-methyl-4-phenoxybutanoate (6n)

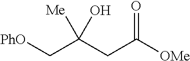 [SMILES: COC(=O)CC(C)(O)COc1ccccc1]

Following general procedure D, 6n was produced as a colorless oil (33.3 mg, 96%). ¹H NMR (500 MHz, CDCl₃): δ 7.30-7.27 (m, 2H), 6.96 (app. t, 1H, J=7.4 Hz), 6.90 (app. d, 2H, J=7.9 Hz), 3.92 (d, 1H, J=8.9 Hz), 3.87 (d, 1H, J=8.9 Hz), 3.78 (s, 1H), 3.71 (s, 3H), 2.78 (d, 1H, J=15.8 Hz), 2.63 (d, 1H, J=15.8 Hz), 1.39 (s, 3H); ¹³C NMR (125 MHz, CDCl₃): δ 173.05, 158.66, 129.64, 121.30, 114.71, 74.20, 70.76, 51.93, 42.16, 25.02; HRMS (DART): Calcd for C₁₂H₁₆O₄ [M+H]⁺: 225.11214. Found: 225.11216.

### Methyl 5-((tert-butyldimethylsilyl)oxy)-3-hydroxy-3-methylpentanoate

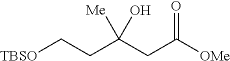 [SMILES: COC(=O)CC(C)(O)CCO[Si](C)(C)C(C)(C)C]

Following general procedure D, 6q was produced as a colorless oil (85.5 mg, 98%). ¹H NMR (500 MHz, CDCl₃): δ 4.20 (s, 1H), 3.88 (app. t, 2H), 3.69 (s, 1H), 2.61 (d, 2H, J=14.9 Hz), 2.55 (d, 2H, J=14.9 Hz), 1.89-1.78 (m, 2H), 1.31 (s, 3H), 0.90 (s, 9H), 0.08 (s, 6H); ¹³C NMR (125 MHz, CDCl₃): δ 172.64, 71.43, 60.43, 51.69, 45.88, 42.01, 27.17, 26.00, 18.25, −5.41; HRMS (DART): Calcd for C₁₃H₂SO₄Si [M+H]⁺: 277.18296. Found: 277.18240. We also synthesized this aldol product from enantiopure lactone, but we were not able to resolve enantiomers of a racemic sample using chiral HPLC of GC to prove that enantiopurity was maintained. Based on the optical activity of 6r and the fact that the ring-opening of (R)-3n proceeds with complete retention of stereochemistry, we propose that this methanolysis also proceeds in >99:1 e.r.

### 1-(Tert-butyl) 4-methyl 2-hydroxy-2-methylsuccinate (6s)

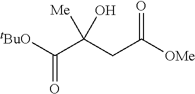 [SMILES: COC(=O)CC(C)(O)C(=O)OC(C)(C)C]

Following general procedure D, 6s was produced as a colorless oil (23.2 mg, 85%). ¹H NMR (500 MHz, CDCl₃): δ 3.69 (s, 1H), 3.68 (s, 3H), 2.90 (d, 1H, J=16.1 Hz), 2.65 (d, 1H, J=16.1 Hz), 1.49 (s, 9H), 1.40 (s, 3H); ¹³C NMR (125 MHz, CDCl₃): δ 174.83, 171.25, 82.64, 72.52, 51.83, 44.29, 27.96, 26.63; HRMS (DART): Calcd for C₁₀H₁₈O₅ [M+H]⁺: 219.12270. Found: 219.12292.

### (R)-Methyl 3-((tert-butyldimethylsilyl)oxy)-3-methyl-5-phenylpentanoate ((R)-6m-TBS)

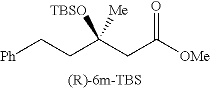 [SMILES: COC(=O)C[C@@](C)(CCc1ccccc1)O[Si](C)(C)C(C)(C)C]

The title compound was synthesized using a procedure adapted from the literature. To a solution of 6m in DCM (0.3M) at 0° C. under a nitrogen atmosphere was added 2,6-lutidine (4.0 eq.) and tert-butyldimethylsilyl trifluoromethanesulfonate (3 eq.). The reaction was allowed to stir at 22° C. and was monitored by ¹H NMR until starting material was entirely consumed (˜5 min). The solution was washed with 1.0 M HCl to remove 2,6-lutidine, and the aqueous layer was extracted with DCM (3×). Organic layers were combined, dried with MgSO₄, and concentrated. The crude product was loaded with hexanes onto a column with increasing eluent polarity (100% hexanes, 19:1 hexanes:Et₂O). 6m-TBS was produced as a colorless oil (for enantioenriched: 29.9 mg, 69%; for rac: 40.8 mg, 72%). Analytical data match literature values. ¹H NMR (500 MHz, CDCl₃): δ 7.29-7.26 (m, 2H), 7.20-7.16 (m, 3H), 3.66 (s, 3H), 2.76-2.65 (m, 2H), 2.57 (s, 2H), 1.89 (d, 1H, J=8.7 Hz), 1.87 (d, 1H, J=8.7 Hz), 1.42 (s, 3H), 0.89 (s, 9H), 0.12 (s, 3H), 0.12 (s, 3H); ¹³C NMR (125 MHz, CDCl₃): δ 171.55, 142.74, 128.52, 125.83, 74.59, 51.51, 46.84, 45.02, 30.80, 28.13, 25.93, 18.33, −1.84, −1.92; HRMS (DART): Calcd for C₁₉H₃₂O₃Si [M+H]⁺: 337.21935. Found: 337.21958. specific rotation: [α]_{D}²⁰ −6.67 (c=0.51, CHCl₃). Comparing to literature data of an enantiomerically enriched sample of 95:5 e.r. ([α]_{D}²⁵ −4.28 (c=0.27, CHCl₃)), the absolute configuration of 6m-TBS was determined to be the (R)-stereoisomer.

Although the present disclosure has been described with respect to one or more particular embodiments and/or examples, it will be understood that other embodiments and/or examples of the present disclosure may be made without departing from the scope of the present disclosure.

1. A method of carbonylating a cyclic substrate comprising:
providing a reaction mixture comprising a cyclic substrate, a solvent, carbon monoxide, and a [LA⁺][Co(CO)₄⁻] catalyst and [LA⁺] is a Lewis acid capable of coordinating to the cyclic substrate;
pressurizing the reaction mixture,
wherein the cyclic substrate is carbonylated.

2. The method of claim 1, wherein the [LA⁺][Co(CO)₄⁻] catalyst has the following structure: 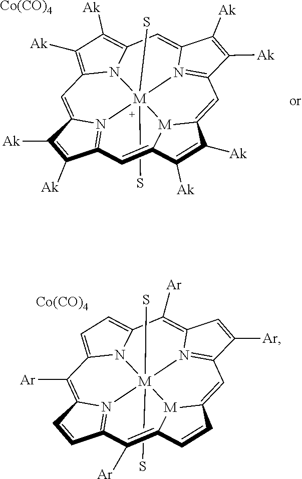
wherein S is solvent, M is a metal chosen from aluminum and chromium, Ak is a substituted linear or branched C₁ to C₁₀ alkyl group or unsubstituted linear or branched C₁ to C₁₀ alkyl group, and Ar is a substituted or unsubstituted aryl group.

3. The method of claim 2, wherein Ar is chosen from phenyl, p-ClC₆H₄, p-FC₆H₄, p-OMeC₆H₄, and 2,4,6-Me₃C₆H₂.

4. The method of claim 2, wherein the [LA⁺][Co(CO)₄⁻] catalyst is chosen from Bis(tetrahydrofuran)-meso-tetraphenylporphyrinato aluminum, [(TPP)Al(THF)₂]⁺, Bis(tetrahydrofuran)-meso-tetra(4-chlorophenyl)porphyrinato aluminum, [(4-ClTPP)Al(THF)₂]⁺, Bis(tetrahydrofuran)-meso-tetra(4-methoxyphenyl)porphyrinato aluminum, [(4-OMeTPP)Al(THF)₂]⁺, Bis(tetrahydrofuran)-meso-tetra(2,4,6-trimethylphenyl)porphyrinato aluminum, [(2,4,6-trimethylTPP)Al(THF)₂]⁺, and Bis(tetrahydrofuran)-octaethylporphyrinato aluminum, [(OEP)Al(THF)₂]⁺.

5. The method of claim 1, wherein the [LA⁺][Co(CO)₄⁻] catalyst has the following structure: 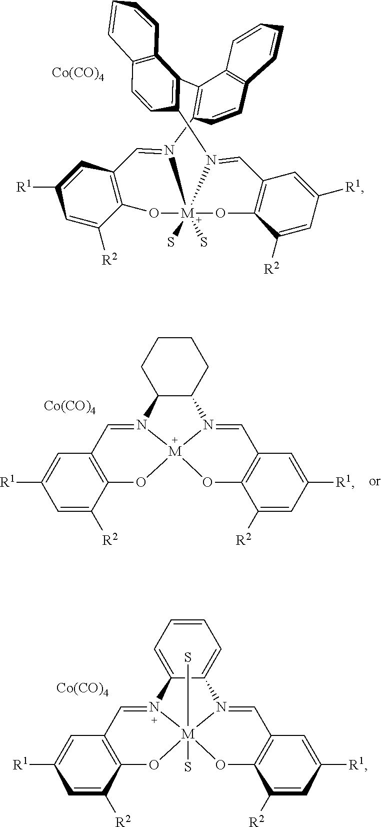
wherein S is solvent, M is a metal chosen from aluminum and chromium, and R¹ and R² are independently chosen from H, alkyl groups, halogen groups, and alkoxide groups.

6. The method of claim 5, wherein the [LA⁺][Co(CO)₄⁻] catalyst is N,N′-Bis(3,5-di-tert-butylsalicylidene)-1,2-phenylenediaminoaluminum, [(salen)Al(THF)₂]⁺.

7. The method of claim 1, wherein the carbonylated cyclic product has a regioselectivity of at least 90:10.

8. The method of claim 1, wherein a mixture of products is formed and the mixture of products comprises a major product and one or more minor product(s), and the major product is the β,β-disubstituted lactone.

9. The method of claim 8, wherein the one or more minor product(s) comprise an α,α-disubstituted lactone.

10. The method of claim 8, wherein 10% or less of the mixture of products comprises the one or more minor product(s).

11. The method of claim 1, further comprising contacting the β,β-disubstituted lactone with a base and, optionally, isolating a β-hydroxy ester.

12. The method of claim 11, wherein the base is an alkoxide base.

13. The method of claim 12, wherein the alkoxide base is chosen from sodium methoxide, sodium ethoxide, sodium n-propoxide, sodium isopropoxide, and sodium tert-butoxide.

14. The method of claim 1, wherein the pressurizing is performed via charging with carbon monoxide.

15. The method of claim 14, wherein the pressurizing is continuously charging with a stream of carbon monoxide or charging to a static environment of carbon monoxide.

16. The method of claim 1, wherein the cyclic substrate is a 2,2,-disubstituted epoxide.

17. The method of claim 16, wherein the 2,2-disubstituted epoxide is chosen from: 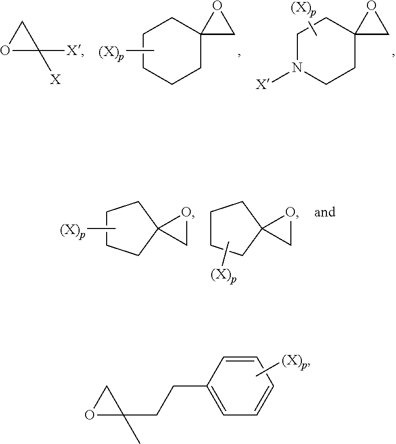
wherein X and X′ at each occurrence is independently chosen from H, alkoxy, alkyl, aryl groups, halides, amino groups, alkenyl groups, alkynyl groups, acyl groups, nitro groups, nitrile groups, hydroxyl groups, protected hydroxyl groups, ether groups, ester groups, thioester groups, and thioether groups, and p is 0-10.

18. The method of claim 17, wherein the 2,2-disubstituted epoxide is chosen from: 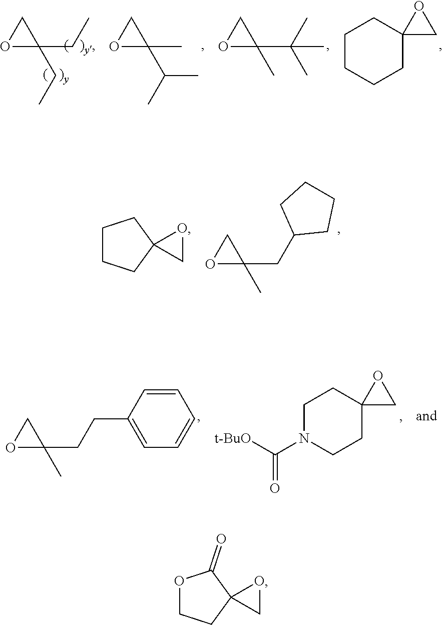
wherein y and y′ are independently 0-40.

19. The method of claim 1, wherein the solvent is chosen from tetrahydrofuran, 2-methyltetrahydrofuran, 2,5-dimethyltetrahydrofuran, diethylether, diisopropyl ether, benzene, toluene, 1,4-dioxane, and combinations thereof.

20. The method of claim 1, wherein the carbon monoxide pressure is 1 atm to 100 atm.

21. The method of claim 1, wherein the catalyst is at a concentration of 0.0001 to 10 mol % relative to the cyclic substrate.

22. The method of claim 1, wherein the reaction mixture is at a temperature of −50 to 120° C.

23. The method of claim 1, further comprising isolating a carbonylated cyclic product from the reaction mixture, wherein the carbonylated cyclic product is a β,β-disubstituted lactone.
